(19) United States
(12) Patent Application Publication
(10) Pub. No.: US 2009/0215908 A1
(43) Pub. Date: Aug. 27, 2009

(54) TOLL LIKE RECEPTOR (TLR) SIGNALING ANTAGONIST

(75) Inventors: Shakti Upadhyay, Navi Mumbai (IN); Yogesh Kanekar, Navi Mumbai (IN); Rajendra Kshirsagar, Navi Mumbai (IN); Vikram Rajagopal, Navi Mumbai (IN); Praneel Datla, Navi Mumbai (IN); Akshaya Bellary, Navi Mumbai (IN); Shiva Prasad Singh, Navi Mumbai (IN)

Correspondence Address: MINTZ, LEVIN, COHN, FERRIS, GLOVSKY AND POPEO, P.C, 5 Palo Alto Square - 6th Floor, 3000 El Camino Real, PALO ALTO, CA, 94306-2155, US

(73) Assignee: RELIANCE LIFE SCIENCES PVT. LTD., Navi Mumbai (IN)

(21) Appl. No.: 12/236,402

(22) Filed: Sep. 23, 2008

(30) Foreign Application Priority Data
Sep. 24, 2007 (IN) 1853/MUM/2007
Dec. 24, 2007 (IN) 2541/MUM/2007

(51) Int. Cl. A61K 31/09; C07C 43/295; C12N 5/06; A61P 35/00; C12Q 1/02

(52) U.S. Cl. 514/719; 568/633; 435/375; 435/29

(57) ABSTRACT

The present invention relates to novel synthetic toll like receptor antagonist. The present invention in particular provides compounds, methods and compositions for specifically inhibiting immune stimulation involving TLR ligands, especially TLR-4. The compounds are potentially useful in treatment of inflammation, autoimmunity, allergy, asthma, graft rejection, graft versus host disease, infection, sepsis, cancer and immunodeficiency.

### CROSS-REFERENCE TO RELATED PATENT APPLICATIONS

The present application claims benefit of the filing date of Indian Provisional Patent Application No. 1853/MUM/2007 filed Sep. 24, 2007, and Indian Provisional Patent Application No. 2541/MUM/2007 filed Dec. 24, 2007 which are incorporated herein by reference in their entireties.

### FIELD OF THE INVENTION

The present invention relates to novel molecules, compositions and methods for preparation and modulation of immune functions mediated through Toll-like receptor (TLR) signaling.

### BACKGROUND OF THE INVENTION

The innate or natural immune system recognizes a wide spectrum of pathogens without a need for prior exposure. Cells of the innate immune system effectively prevent free growth of bacteria within the body; however, many pathogens have evolved mechanisms allowing them to evade the innate immune system, but unlike the adaptive immune system, it does not confer long-lasting or protective immunity to the host. Innate immune systems provide immediate defense against infection, and are found in all classes of plant and animal life. The main cells responsible for innate immunity are monocytes/macrophages and neutrophils, which phagocytose microbial pathogens and are responsible for triggering the innate, inflammatory, and specific immune responses.

Toll-like receptor are a family of receptors involved in the recognition of a wide range of microbial molecules e.g. Lipopolysaccharides (LPS) from Gram-negative bacteria and peptidoglycan from Gram-positive bacteria. The prototype receptor Toll was first identified in the fruit fly Drosophila but several TLR was found in mammals, particularly on mononuclear phagocytes. Toll-like receptors (TLRs) are a class of single membrane-spanning non-catalytic which are designated TLR2, TLR 4, TLR5 and each receptor recognizes a small range of structurally conserved molecules once they have breached physical barriers such as the skin or intestinal tract mucosa, and activate immune cell responses. They are believed to play a key role in the innate immune system. TLRs are a type of pattern recognition receptors (PRRs) and recognize molecules that are broadly shared by pathogens but distinguishable from host molecules, collectively referred to as pathogen-associated molecular patterns (PAMPs).

The discovery of the Toll-like receptors finally identified the innate immune receptors that were responsible for many of the innate immune functions that had been studied for many years. Interestingly, TLRs seem only to be involved in the cytokine production and cellular activation in response to microbes, and do not play a significant role in the adhesion and phagocytosis of microorganisms. Binding of TLR leads to the production of inflammatory cytokines, including TNF-alpha and IL-12 and enhances the cells' antimicrobial killing mechanisms and antigen presenting capacity. The function of the TLRs was discovered by Beutler and colleagues. (Poltorak A, He X, Smirnova I, Liu M Y, Van Huffel C, Du X, Birdwell D, Alejos E, Silva M, Galanos C, Freudenberg M, Ricciardi-Castagnoli P, Layton B, Beutler B. Defective LPS signaling in C3H/HeJ and C57BL/10ScCr mice: mutations in Tlr4 gene. Science. 1998; 282: 2085-88.) These workers used positional cloning to prove that mice that could not respond to LPS had mutations that abolished the function of TLR4. This identified TLR4 as a key component of the receptor for LPS, and strongly suggested that other Toll-like receptors might detect other signature molecules of microbes, such as those mentioned above.

The chemical structure and the molecular basis of the recognition of LPS by serum proteins has gained attention in recent years, which has led to the discovery of a family of receptors like Toll like receptors. It has been estimated that most mammalian species have between ten and fifteen types of Toll-like receptors. Thirteen TLRs (named simply TLR1 to TLR13) have been identified in humans and mice together, and equivalent forms of many of these have been found in other mammalian species (Du, X., Poltorak, A., Wei, Y., and Beutler, B. Three novel mammalian toll-like receptors: gene structure, expression, and evolution. Eur. Cytokine Netw. 2000; 11: 362-71; Chuang, T. H., and Ulevitch, R. J. Cloning and characterization of a sub-family of human toll-like receptors: hTLR7, hTLR8 and hTLR9. Eur. Cytokine Netw. 2000; 11:372-78; Tabeta, K., Georgel, P., Janssen, E., Du, X., Hoebe, K., Crozat, K., Mudd, S., Shamel, L., Sovath, S., Goode, J. et al. Toll-like receptors 9 and 3 as essential components of innate immune defense against mouse cytomegalovirus infection. Proc. Narl Acad. Sci. U. S. A. 2004; 101: 3516-21.).

The significance of the toll like receptors in the immune response to LPS has further demonstrated specifically two receptors TLR2 and TLR4 (Yang R B, Mark M R, Gray A, Huang A, Xie M H, Zhang M, Goddard A, Wood W I, Gurney A L, Godowski P J. Toll-like receptor-2 mediates lipopolysaccharide-induced cellular signalling. Nature. 1998; 395: 284-88; Kirschning C J, Wesche H, Merrill Ayres T, Rothe M. Human toll-like receptor 2 confers responsiveness to bacterial lipopolysaccharide. J Exp Med. 1998; 188: 2091-97.). Further reports concluded that TLR4 was required for a response to LPS. (Poltorak A, et al., Science. 1998; 282: 2085-88; Qureshi S T, Larivière L, Leveque G, Clermont S, Moore K J, Gros P, Malo D. Endotoxin-tolerant mice have mutations in Toll-like receptor 4(Tlr4) J Exp Med. 1999; 189: 615-25).

Although LPS is an immunomodulatory agent, its medicinal use is limited due to its extreme toxicity including the induction of systemic inflammatory response syndrome. The biologically active endotoxio endotoxic sub-structural moeity of LPS is a lipid-A, a phosphorylated, multiple fatty acid acylated glucosamine disaccharide that serves to anchor the entire structure in the outer membrane of the gram-negative bacteria. The toxic effects of the lipid A was addressed by selective chemical modification of the lipid A to produce monophosphoryl lipid A compounds (MPL®: vaccine adjuvant and immunostimulant from Corixa (Seattle, Wash., US) and structurally like MPL® compounds) which is described in U.S. Pat. Nos. 4,436,727; 4,877,611; 4,866,034; 4,912,094; 4,987,237; and Johnson et. al. (Johnson D A, Keegan D S, Sowell C G, Livesay M T, Johnson C L, Taubner L M, Harris A, Myers K R, Thompson J D, Gustafson G L, Rhodes M J, Ulrich J T, Ward J R, Yorgensen Y M, Cantrell J L, Brookshire V G. 3-O-Desacyl monophosphoryl lipid A derivatives: synthesis and immunostimulant activities. J Med Chem. 1999; 42: 4640-49).

US Pub. patent App. 20070167409 is based on discovery that animals that do not express Toll-like receptor 2 (TLR2) are protected from dextran sulfate sodium (DSS) induction of colitis, a model for inflammatory bowel disease (IBD). The invention relates to the agents that block activation of TLR2 to treat or to prevent colitis and related diseases or conditions, as well as other diseases or conditions characterized by activation of TLR2.

The European patent EP1635846 has methods and compositions useful for modulating signaling through Toll-like receptors that involve contacting a TLR-expressing cell with a small molecule having a core structure including at least two rings. Certain of the compounds are 4-primary amino quinolines.

US Pub. patent App. US20060058365 relates to the treatment of inflammatory bowel disease (IBD) and related gastrointestinal pathologies that are cytokine-mediated or associated with Toll-like receptor 4 using methimazole derivatives and tautomeric cyclic thiones. Further US Pub. patent App. 20050004144 provides a broad-spectrum, long-lasting, and non-toxic combination of synthetic immunostimulatory agents, which are useful for activating the immune system of a mammal and treating diseases such as cancer and autoimmune disease involving 7-substituted, 8-substituted and 7,8-di substituted 7-deazaguanosines.

US Pub. patent App. 20030139364 involves administration of an imidazoquinoline agent in combination with another therapeutic agent in synergistic amounts to enhance ADCC, stimulate immune responses and/or patient and treat certain disorders.

Different phenanthrene derivatives that have been used in the prior art are discussed below:

WO 2006027345 discloses novel 3-Thia-10-aza-phenanthrene derivatives as novel effective PDE4 inhibitors.

WO2006089881 has described novel phenanthrene derivatives as antiinflammatory agents and WO 9113855 has provided a new phenanthrene derivative having IL-1 inhibiting activity and useful for the treatment of chronic inflammatory diseases.

U.S. Pat. No. 3,683,091 specifically provides di-7-hydroxy or methyl-2,3,4,4a,9,10-hexahydrophenanthren-2-one and 4a-alkyl derivatives, useful as specific anti-acne agents.

GB1069067 provides novel phenanthrene derivatives having analgesic and morphine antagonistic activity.

U.S. Pat. No. 476,678 discloses phenanthrene derivatives possessing valuable fungicidal properties useful in agriculture, horticulture and other antifungal compositions.

U.S. Pat. No. 4,808,625 discloses aminoalkanol derivatives of containing a polycarbocyclic aromatic ring system such as phenanthrene, as biocidal agents, particularly antitumor agents.

JP 8067626 discloses hydrogenated condensed ring hydrocarbons such as hydrogenated phenanthrene (e.g. 1,2,3,4,5,6,7,8-octahydrophenanthrene), a hydrogenated anthracene (e.g. 1,2-dihydroanthracene), a hydrogenated naphthalene (e.g. 1,2-dihydronaphthalene), etc as capable of inhibiting the carcinogenesis induced by a carcinogenic organic compound without accompanied by side effects

GB2186570 provides 9,10-dihydrophenanthrene derivatives which are useful in treating diseases characterized by an immunological imbalance and bacterial and viral infections in mammals.

Certain phenanthrene derivatives from plants have also been used in the prior art for immune system disorders such as 9,10-dihydrophenanthrene called eulophiol isolated from the tubers of Eulophia nuda (Bhandari S R and Kapadi A H. A 9,10-dihydrophenanthrene from tubers of Eulophia nuda; Phytochemistry. 1983; 22: 747-48; Tuchinda P, Udchachon J, Khumtaveeporn K, Taylor W C, Engelhardt L M, White, A. H. Phenanthrenes of Eulophia nuda. Phytochemistry. 1988; 27: 3267-71; Majumder P L, Sen S, Majumder S. Phenanthrene derivatives from the orchid Coelogyne cristata. Phytochemistry. 2001; 58: 581-586).

JP 7267895 has provided plant extracts containing phenanthrene derivatives from Raikoutou (a root or leaf of Tripterygium wilfordii Hook. F.) a Chinese herbal drug. These compounds have been found to be useful as a therapeutic agent for diseases owing to leukotriene of pollinosis, bronchial asthma, arthritis etc.

A number of phenanthrene derivatives have been extracted as a principal constituent from plant species Orchidaceae family has been reported, (Majumder P L and Sen R C. Pendulin, a polyoxygenated phenanthrene derivative from the Orchidaceae Cymbidum pendulum. Phytochemistry. 1991; 30: 2432-2434; Shimizu M, Shogawa H, Hayashi T, Arisawa M, Suzuki S, Yoshizaki M, Morita N, Ferro E, Basualdo I, Berganza L H. Anti-inflammatory constituents of topically applied crude drugs. III. Constituents and anti-inflammatory effect of Paraguayan crude drug “Tamandá cuná” (Catasetum barbatum LINDLE). Chem Pharm Bull (Tokyo). 1988; 36: 4447-52.). These extracts have been used as antipyretic, antioxidant and antispasmolytic agents.

WO 2006089881 has focused on methoxy phenanthrene derivatives from Tamus communis as anti-inflammatory agents.

The past decade has seen an explosion in TLR antagonist research, including their potential implication in auto-immune and chronic inflammatory diseases.

### SUMMARY OF THE INVENTION

It is an object of the present invention to provide small molecules for modulation of immune functions through Toll like receptors.

It is an object of this invention to provide small molecules for inhibitions of TLR signaling in response to TLR ligands.

It is an object of the present invention to provide phenanthrene derivatives and its analogs for modulation of the immune functions through toll like receptors.

It is an object of the present invention to provide methods of preparation or isolation of these phenanthrene derivatives and its analogs.

It is an object of the present invention to provide compositions of the phenanthrene derivatives and its analogs.

It is an object of the present invention to provide compositions useful for the prevention or treatment of inflammation, wounds, autoimmunity, allergy, asthma, graft rejection, graft versus host disease, infection, sepsis, cancer and immunodeficiency.

It is also the object of this invention to provide a method and composition for affecting the TLR mediated signaling in response to a TLR ligand.

It is an object of the present invention to provide phenanthrene based molecules for inhibition of TLR ligand, which can be used in combinations with other agents.

The inventors of the present invention have designed novel phenanthrene derivatives that act as antagonists of Toll-like receptors and methods and compositions for modulating the immune functions through Toll-like receptor. The novel phenanthrene derivatives of the present invention have found their potential in inhibiting signaling of Toll-like receptors.

The present invention focuses on novel derivatives of phenanthrenes for potential use in inhibition of immune stimulation involving toll like receptor ligands. These molecules have been developed for potential use in treatment of inflammation, autoimmunity, allergy, asthma, graft rejection, graft versus host disease, infection, sepsis, cancer and immunodeficiency. More specifically, whereas the agents described herein have been discovered to affect TLRs directly and thus directly affect TLR-bearing cells, e.g., antigen-presenting cells (APCs), such agents can be used in conjunction with additional agents which affect non-APC immune cells, e.g., T lymphocytes (T cells). Such an approach effectively introduces an immunomodulatory intervention at two levels: innate immunity and acquired immunity.

The present invention relates to phenanthrene derivatives, methods of their preparation, and compositions for use in TLR mediated immune conditions. The present invention also relates to compositions and methods for modulating immune functions mediated through Toll-like receptor (TLR) molecules.

In one embodiment the present invention provides compositions that are useful for the prevention or treatment of inflammation, wounds, autoimmunity, allergy, asthma, graft rejection, graft versus host disease, infection, sepsis, cancer, and immunodeficiency. In the preferred embodiments the compositions as described in the present invention or the compositions are useful as for inhibition of TLR signaling in response to TLR ligands.

In the preferred embodiments the compositions for inhibition of TLR signaling as described in the present invention in a therapeutically effective amount and pharmaceutically inert adjuvants, diluents or carriers.

In one embodiment the compositions as described in the present invention or composition and the method of manufacture comprising the same is believed to have the ability to inhibit TLR signaling under physiological conditions, and thereby would have corresponding effectiveness for prevention or treatment of inflammation, wounds, autoimmunity, allergy, asthma, graft rejection, graft versus host disease, infection, sepsis, cancer, and immunodeficiency.

In yet other embodiments, the compositions as described in the present invention can be used to prevent or treat clinical manifestations and diseases caused by microbial pathogens.

In yet other embodiments on the basis of inhibition of TLR signaling, the composition can be used in veterinary medicine to prevent or treat clinical manifestations and diseases caused by microbial pathogens.

In preferred embodiments the present invention also provides pharmaceutical formulations either by itself or in a suitable pharmaceutically acceptable adjuvant useful for inhibition of TLR mediated clinical manifestations.

As a feature of the present invention, the methods of the invention can be combined with administration of additional agents to achieve synergistic effect on TLR-mediated immunostimulation. More specifically, whereas the agents described herein have been discovered to affect TLRs directly and thus directly affect TLR-bearing cells, e.g., antigen-presenting cells (APCs), such agents can be used in conjunction with additional agents which affect non-APC immune cells, e.g., T lymphocytes (T cells). Such an approach effectively introduces an immunomodulatory intervention at two levels: innate immunity and acquired immunity. Since innate immunity is believed to initiate and support acquired immunity, the combination intervention is synergistic

In another embodiment of the invention, a method of affecting TLR-mediated signaling in response to a TLR ligand is provided.

In one embodiment of the invention, a method of inhibiting TLR-mediated immunostimulatory signaling is provided.

In another embodiment, the invention provides a method of affecting TLR-mediated immunostimulation in a subject.

Methods of treatment for variety of conditions involving autoimmunity, inflammation, allergy, asthma, graft rejection, graft-versus-host disease (GvHD), infection, sepsis, cancer, and immunodeficiency. Generally, methods useful in the treatment of conditions involving infection, cancer, and immunodeficiency will employ small molecules that augment TLR-mediated signaling in response to a suitable TLR ligand. In some instances the methods can be used to inhibit or promote TLR-mediated signaling in response to a TLR ligand or TLR signaling agonist. In some instances the methods can be used to inhibit TLR-mediated immunostimulatory signaling in response to a TLR ligand or TLR signaling agonist. In some instances the methods can be used to inhibit or promote TLR-mediated immunostimulation in a subject. In some instances the methods can be used to inhibit TLR-mediated immunostimulation in a subject. In some instances the methods can be used to inhibit an immunostimulatory nucleic acid-associated response in a subject. In one embodiment, the present invention provides molecules and methods useful for modulating TLR-mediated signaling. The molecules of the present invention are applicable to alter any TLR-mediated signaling in response to a suitable ligand or signaling agonist.

In one embodiment the present invention also provides methods for identifying agents that decrease or inhibit activation of Toll-like receptor 2. These methods involve (i) contacting a cell expressing the receptor with a candidate agent in the presence of an activator of the receptor (in vitro or in vivo) and (ii) determining the effect of the agent on activation of the receptor. Detection of a decrease in activation of the receptor by the activator in the presence of the agent indicates the identification of agent that can be used to decrease or inhibit activation of the receptor. In these methods, the effect of the agent on the activation of the receptor can be determined by analysis of the expression of a reporter gene that is under the control of a promoter that is induced in a signaling pathway triggered by activation of the receptor.

In one aspect, the present invention provides compounds which can be isolated from plant species such as Eulophia. Preferably this base compound Eulophiol (RSCL-0520) is extracted and further derivatives can be prepared by synthetic routes.

In another aspect the present invention also provides compounds, which can be prepared by synthetic routes.

In one aspect of the invention, a method of affecting TLR-mediated signaling in response to a TLR ligand is provided. The method according to this aspect involves contacting a cell expressing a TLR with an effective amount of a compound of Formula I

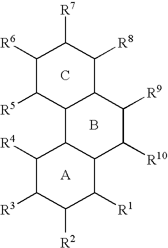

in which R¹, R², R³, R⁴, R⁵, R⁶, R⁷, R⁸, R⁹ and R¹⁰ are identical or different and may each be hydrogen, hydroxy, C₁₋₆ alkoxy, C₁₋₆ alkyl, halogen, haloalkyl, acyloxy, hydroxyalkyl, alkenyl, alkenyloxy, carboxyl, carbalkoxy, carbamido, a conjugated group, substituted or unsubstituted phenyl, substituted or unsubstituted heterocyclic group, nitro, amino, acylamino, dialkylamino, nitric oxide (NO)-releasing moiety, pharmaceutically acceptable salts, amides and esters thereof. Ring A, Ring B, and Ring C may be aliphatic or aromatic.

Some compounds of the present invention but not limited to the above general formula are listed below:
2,7-dihydroxy-3,4-dimethoxyphenanthrene (RSCL-0518)
2,7-diacetoxy-3,4-dimethoxyphenanthrene (RSCL-0519)
2,3,4,7-tetramethoxyphenanthrene (RSCL-0575)
1,5-dihydroxy-2,7-dimethoxy-9,10-dihydrophenanthrene (RSCL-0521)
1,5-diacetoxy-2,7-dimethoxy-9,10-dihydrophenanthrene (RSCL 0520)
3,4-dimethoxyphenanthrene-2,7-bis-[(2E)-3-[3,4-bis(acetyloxy)phenyl]acrylate (RSCL-0522)
1,5-dibenzyloxy-2,7-dimethoxy phenanthrene (RSCL-0638)

The present invention and other objects, features, and advantages of the present invention will become further apparent in the following Detailed Description of the Invention and the accompanying Figures and embodiments.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein.

FIG. 1a shows that RSCL-0520 inhibits TLR4 induced TNF-α secretion in human monocytic (THP-1) cells.

FIG. 1b shows that RSCL-0520 inhibits TLR2 and TLR4 induced TNF-α secretion in and peripheral blood monocytes (PBMCs).

FIG. 2a shows inhibition of TNF-α secretion in THP-1 cells by RSCL-0520 is dose-dependent

FIG. 2b indicates that RSCL-0520 is not toxic to THP-1 cells

FIG. 3 shows RSCL-0520 inhibits LPS induced TNF-α release in PBMC

FIG. 4 shows the dose dependent effect of RSCL-0520 on different concentrations of LPS in THP-1 cells

FIG. 5a shows effect of RSCL-0520 on TNF-α mRNA expression in THP-1 cells in real time

FIG. 5b shows effect of RSCL-0520 on mRNA expression of pro-inflammatory genes in THP-1 Cells

FIG. 5c Demonstrates ability of RSCL-0520 to inhibit Arachidonic acid induced PGE2 release in A549 cells

FIG. 6 shows RSCL-0520 suppresses LPS induced Nitric oxide (NO) release in RAW 264.7 cells

FIG. 7a represents effect of RSCL-0520 on activation of NEMO and degradation of I kappa B-alpha (IκB-α).

FIG. 7b represents effect of RSCL-0520 activation of NF-κB.

FIG. 7c represents effect of RSCL-0520 on translocation of NF-κB to the nucleus

FIG. 8a represents effect of RSCL-0520 on TLR related genes

FIG. 8b represents RSCL-0520 inhibits MyD88 dependent TLR signaling by LPS.

FIG. 9a shows pre-treatment of RSCL-0520 suppresses LPS induced TNF-α release in Balb/c mice

FIG. 9b Treatment with RSCL-0520 post LPS induction suppresses the induced TNF-α release in Balb/c mice

### DETAILED DESCRIPTION OF THE INVENTION

### Definitions

The term “synthetic small molecules” as used herein refers to molecules with basic phenanthrene backbone.

The term “LPS” as used herein refers to Lipopolysaccharide. LPS, which is contained in the outer membrane of the cell wall of various gram-negative bacteria, consists of a glycolipid called “Lipid A” to which various saccharides are bonded. It has been known for along time that LPS is the main component of endotoxins.

The term “TLR” as used herein refers to Toll like receptor.

The term “pharmaceutically acceptable salt” as use herein, refers to those salts which are, within the scope of sound medical judgement, suitable for use in contact with the tissues of humans and animals without undue toxicity, irritation, allergic response and the like and are commensurate with a reasonable benefit/risk ratio. Pharmaceutically acceptable salts are well known in the art. For example, S. M. Berge et al. describes pharmaceutically acceptable salts in detail. (Berge S M, Bighley L D, Monkhouse D C. Pharmaceutical salts. J Pharm Sci. 1977; 66:1-19). The salts can be prepared in situ during the final isolation and purification of a compound of the invention or separately by reacting the free base group with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, suberate, sulfate, tartrate, thiocyanate, toluenesulfonate, undecanoate, valerate salts, and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The term “pharmaceutically acceptable ester,” as used herein, represents esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic, and alkanedioic acids, in which each alkyl or alkenyl group preferably has not more than 6 carbon atoms. Examples of particular esters include formates, acetates, propionates, butyrates, acrylates, and ethylsuccinates. Amides and esters could also be prepared by coupling the compounds of the present invention with phenocli acids such as Non-steroidal anti-inflammatory drugs (NSAIDs) etc.

The term “pharmaceutically acceptable pro-drugs,” as used herein, means pro-drugs of the compounds of the present invention which are, within the scope of sound medical judgement, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “prodrug,” as used herein, represents compounds that are transformed in vivo into a parent compound of the above formula, for example, by hydrolysis in blood. A thorough discussion of prodrugs is provided in T. Higuchi and V. Stella (Pro-drugs as Novel Delivery Systems,” Vol. 14 of the A.C.S. Symposium Series, Edward B. Roche, ed., “Bioreversible Carriers in Drug Design,” American Pharmaceutical Association and Pergamon Press, 1987), and Judkins et al (Synthetic Communications. 1996; 26: 4351-67), each of which is incorporated herein by reference.

Asymmetric or chiral centers may exist in the compounds of the present invention. The present invention includes the various stereoisomers and mixtures thereof. Individual stereoisomers of compounds or the present invention may be prepared synthetically from commercially available starting materials that contain asymmetric or chiral centers or by preparation of mixtures of enantiometic compounds followed by resolution well-known to those of ordinary skill in the art. These methods of resolution are exemplified by (1) attachment of a racemic mixture of enantiomers, designated (+/−), to a chiral auxiliary, separation of the resulting diastereomers by recrystallization or chromatography and liberation of the optically pure product from the auxiliary or (2) direct separation of the mixture of optical enantiomers on chiral chromatographic columns. Enantiomers are designated herein by the symbols “R” or “S,” depending on the configuration of substituents around the chiral carbon atom, or are drawn by conventional means with a bolded line defining a substituent above the plane of the page in three-dimensional space and a hashed or dashed line defining a substituent beneath the plane of the printed page in three-dimensional space. If no stereochemical designation is made, it is to be assumed that the structure definition includes both stereochemical possibilities.

### Compounds of the Present Invention

The present invention relates to the compounds of formula (I) and derivatives thereof including but not limited to polymorphs, isomers and prodrugs thereof, geometric or optical isomers thereof, and pharmaceutically acceptable esters, ethers, carbamates of such compounds, all solvates and hydrates thereof and all salts thereof.

Particularly the present invention provides the compounds of formula I which are represented by structure numbers as follows

A compound represented by the formula (I):

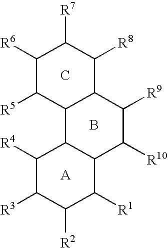

in which R¹, R², R³, R⁴, R⁵, R⁶, R⁷, R¹, R⁹ and R¹⁰ are identical or different and may each be hydrogen, hydroxy, C₁₋₆ alkoxy, C₁₋₆ alkyl, halogen, haloalkyl, acyloxy, hydroxyalkyl, alkenyl, alkenyloxy, carboxyl, carbalkoxy, carbamido, a conjugated group, substituted or unsubstituted phenyl, substituted or unsubstituted heterocyclic group, nitro, amino, acylamino, dialkylamino, nitric oxide (NO)-releasing moiety, pharmaceutically acceptable salts, amides and esters thereof. Ring A, Ring B, and Ring C may be aliphatic or aromatic.

The compounds of the present invention may contain asymmetric or chiral centers, and therefore may exist in different stereoisomeric forms. All suitable optical isomers and stereoisomeric forms of the compounds of the present invention as well as mixtures thereof, including racemic mixtures, form part of the present invention. In addition, the present invention embraces all geometric and positional isomers. Moreover, some compounds of the present invention may exhibit polymorphism. The present invention includes all polymorphic forms of the compounds according to the invention, which forms the further aspect of the invention. It is to be understood that the present invention encompasses any and all racemic, optically-active, polymorphic and stereoisomeric forms, or mixtures thereof, which form or forms possess properties useful in the treatment of the conditions indicated herein.

Furthermore, the present invention also include isotopically-labeled compounds of the present invention which are identical to those recited herein, but for the fact that one or more atoms are replaced by an atom having an atomic mass or mass number different from the atomic mass or mass number usually found in nature.

### Preparation of Compounds of the Present Invention:

All of the starting materials used in any of these methods are commercially available from chemical vendors such as Aldrich, Sigma, Nova Biochemicals, Bachem Biosciences, Advanced ChemTech, and the like, or may be readily synthesized by known procedures.

The reaction products are isolated and purified by conventional methods, typically by solvent extraction into a compatible solvent. The products may be further purified by column chromatography or other appropriate methods, including medium pressure or high pressure liquid chromatography.

The compounds and methods of the invention are described in further detail, as follows:

### Synthesis of TLR Antagonists:

A general method of synthesis includes the preparation of phenanthrene molecules by any of the following approaches:

1. A mixture of substituted benzaldehyde, 3,4,5-trimethoxyphenyl acetic acid, acetic anhydride and triethylamine is refluxed and then acidified to obtain acid (A) which is crystallized from suitable solvent. The acid (A) obtained is mixed with quinoline and copper chromite and refluxed under inert atmosphere and then precipitated with ethyl acetate. After filtration, the filtrate is washed with dilute hydrochloric acid, water, and brine, dried (sodium sulphate), and evaporated under reduced pressure to isolate the product. (B) A stirred solution of the product obtained (B) and iodine in ethanol is irradiated at 254 nm. The solution is then evaporated under reduced pressure and the product (C) is isolated by silica gel column chromatography. (Pet-ether-ethyl acetate gradient)

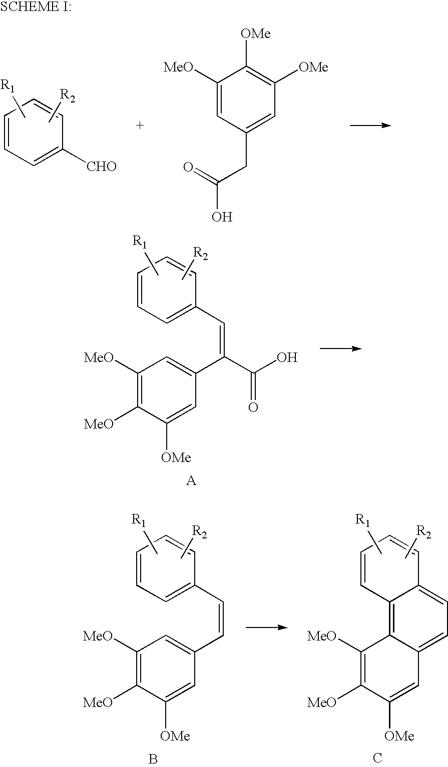

An alternate approach to the above reaction is Suzuki coupling of the appropriate reactants 1 and 2 gives a biaryl dialdehyde 3. The compound 3 on Wittig olefination, and ring closing metathesis of the resultant diene 4 with the Grubbs second-generation ruthenium catalyst gives 5.

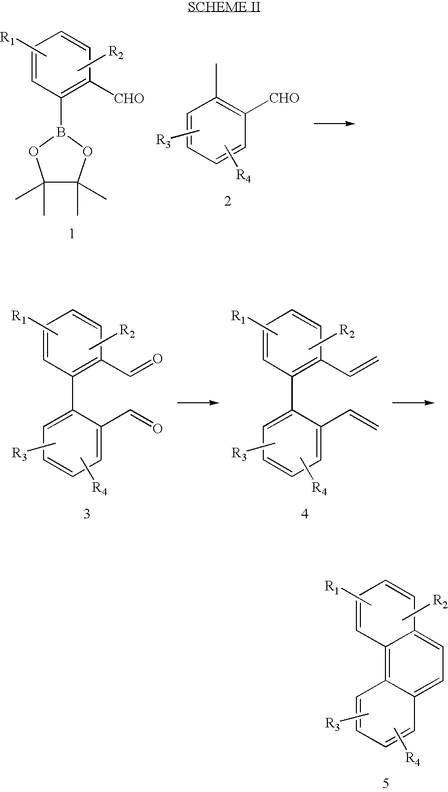

An alternate approach is Pschorr synthesis wherein substituted nitroaldehyde, 3,4,5-trimethoxyphenyl acetic acid, acetic anhydride and triethylamine are refluxed. The mixture is then acidified and stirred. The precipitated solid is filtered off and washed with hot water, leaving acid (A). A solution of the acid (A) in 5N ammonium hydroxide is added to a slurry of iron (II) sulphate, and concentrated ammonium hydroxide at 80-90° C. After heating, the product is filtered and the precipitate is washed with 5N-ammonium hydroxide. The cooled filtrate is acidified with glacial acetic acid and the precipitate is filtered off and vacuum dried (B). To a mixture of amine (B), ethanol and 5N-hydrochloric acid at 0° C. is added a 50% ethanolic solution of isopentyl nitrite. The mixture stirred and then diluted with water, further copper powder is added and stirred at 50° C. The cooled mixture is then extracted with ethyl acetate, which on concentration under reduced pressure gives acid (C). A mixture of acid (C), quinoline and basic copper carbonate is refluxed, and extracted with ethyl acetate. The ethyl acetate layer is washed with acid, alkali, and evaporated. The product (P) is purified by column chromatography on silica (with pet-ether-ethyl acetate gradient).

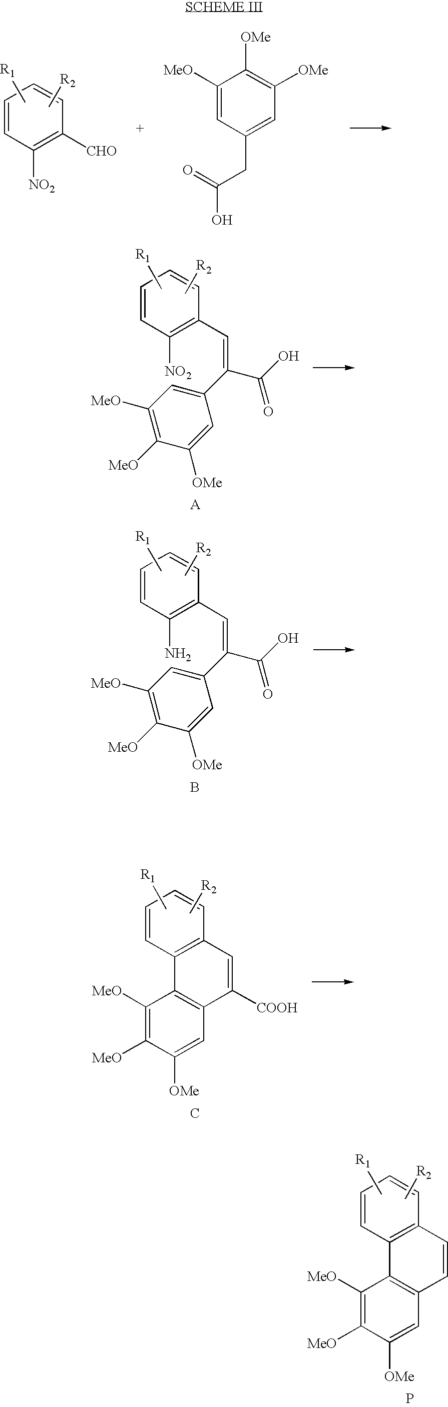

### Therapeutic Use of TLR Antagonist

The present invention provides agents that can be used to prevent or to treat LPS mediated diseases or conditions that are characterized by TLR activation.

The conditions that are prevented or treated but are not limited to inflammatory bowel disease (IBD), sepsis, periodontal disease, mucositis, acne, cardiovascular disease, chronic obstructive pulmonary disease, arthritis, cystic fibrosis, bacterial-induced infections, viral-induced infections, mycoplasma-associated diseases, post herpetic neuralgia, ischemia/reperfusion injury, asthma, stroke, brain injury, necrotizing enterocolitis, bed sores, leprosy, atopic dermatitis, psoriasis, trauma, neurodegenerative disease, amphotericin B-induced fever and nephritis, coronary artery bypass grafting, and atherosclerosis.

### Delivery and Dosage of the TLR Antagonist:

The present invention provides compositions comprising carbohydrate based molecules in an effective amount that achieves the desired therapeutic effect for a particular condition, patient and mode of administration. The dosage level selected depends on the route of administration and the severity of the condition being treated.

For example, for adults, the doses are generally from about 0.01 to about 100 mg/kg, desirably 0.1-1 mg/kg by inhalation, desirably 0.5-10 mg/kg per day by oral administration, and desirably 0.1-1 mg/kg body weight per day by intravenous administration. Doses are determined for each particular case using standard methods in accordance with factors unique to the patient, including age, weight, general state of health, and other factors that can influence the efficacy of the compound(s) of the invention.

Further the administration of the compounds of the present invention is not limited to mammal, including humans, be limited to a particular mode of administration, dosage, or frequency of dosing.

The present invention encompasses all modes of administration, including oral, intraperitoneal, intramuscular, intravenous, intra-articular, intralesional, subcutaneous, or nasally, rectally, buccally, or any other route sufficient to provide a dose adequate to prevent or treat excess or undesired TLR activity.

The present invention also contemplates that one or more compounds may be administered to a mammal in a single dose or multiple doses. When multiple doses are administered, the doses may be separated from one another by, for example, several hours, one day, one week or one month. It is to be understood that, for any particular subject, specific dosage regimes should be adjusted over time according to the individual need and the professional judgment of the person administering or supervising the administration of a pharmaceutical composition that includes a compound of the invention.

The present invention provides compositions of phenanthrene derivatives being TLR antagonists which may be prepared by conventional methods using one or more pharmaceutically acceptable excipients or adjuvants which may comprise inert diluents, sterile aqueous media and/or various non toxic solvents. The pharmaceutically acceptable carrier or diluents may be used as described in literature (The Science and Practice of Pharmacy (20th ed.), ed. A. R. Gennaro, Lippincott Williams & Wilkins, 2000, Philadelphia; Encyclopedia of Pharmaceutical Technology, eds. J. Swarbrick and J. C. Boylan, 1988 1999, Marcel Dekker, New York).

The compositions may be presented in the form of tablets, pills, granules, powders, aqueous solutions or suspensions, injectable solutions, elixirs, or syrups, and the compositions may optionally contain one or more agents chosen from the group comprising sweeteners, flavorings, colorings, and stabilizers in order to obtain pharmaceutically acceptable preparations.

The choice of vehicle and the content of active substance in the vehicle are generally determined in accordance with the solubility and chemical properties of the product, the particular mode of administration, and the provisions to be observed in pharmaceutical practice. For example, excipients such as lactose, sodium citrate, calcium carbonate, and dicalcium phosphate and disintegrating agents such as starch, alginic acids, and certain complex silicates combined with lubricants (e.g., magnesium stearate, sodium lauryl sulfate, and talc) may be used for preparing tablets. To prepare a capsule, it is advantageous to use lactose and high molecular weight polyethylene glycols. When aqueous suspensions are used, they may contain emulsifying agents that facilitate suspension. Diluents such as sucrose, ethanol, polyethylene glycol, propylene glycol, glycerol, chloroform, or mixtures thereof may also be used.

For parenteral administration, emulsions, suspensions, or solutions of the compositions of the invention in vegetable oil (e.g., sesame oil, groundnut oil, or olive oil), aqueous-organic solutions (e.g., water and propylene glycol), injectable organic esters (e.g., ethyl oleate), or sterile aqueous solutions of the pharmaceutically acceptable salts are used. The solutions of the salts of the compositions of the invention are especially useful for administration by intramuscular or subcutaneous injection. Aqueous solutions that include solutions of the salts in pure distilled water may be used for intravenous administration with the proviso that (i) their pH is adjusted suitably, (ii) they are appropriately buffered and rendered isotonic with a sufficient quantity of glucose or sodium chloride, and (iii) they are sterilized by heating, irradiation, or microfiltration. Suitable compositions containing a compound of the invention may be dissolved or suspended in a suitable carrier for use in a nebulizer or a suspension or solution aerosol, or may be absorbed or adsorbed onto a suitable solid carrier for use in a dry powder inhaler. Solid compositions for rectal administration include suppositories formulated in accordance with known methods and containing at least one compound of formula I or II.

Dosage formulations of a compound of the invention to be used for therapeutic administration must be sterile. Sterility is readily accomplished by filtration through sterile membranes (e.g., 0.2 micron membranes) or by other conventional methods. Formulations typically are stored in lyophilized form or as an aqueous solution. The pH of the compositions of this invention is typically between 3 and 11, more desirably between 5 and 9, and most desirably between 7 and 8, inclusive. While a desirable route of administration is by injection such as intravenously (bolus and/or infusion), other methods of administration may be used. For example, compositions may be administered subcutaneously, intramuscularly, colonically, rectally, nasally, or intraperitoneally in a variety of dosage forms such as suppositories, implanted pellets or small cylinders, aerosols, oral dosage formulations, and topical formulations such as ointments, drops, and dermal patches. A compound of the invention is desirably incorporated into shaped articles such as implants, including but not limited to valves, stunts, tubing, and prostheses, which may employ inert materials such as synthetic polymers or silicones, (e.g., Silastic, silicone rubber, or other commercially available polymers). Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, a TLR2 inhibitor of the invention may be coupled to a class of biodegradable polymers useful in achieving controlled release of a drug, for example polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates, and cross linked or amphipathic block copolymers of hydrogels.

A compound of the invention may also be administered in the form of liposome delivery systems, such as small unilamellar vesicles, large unilamellar vesicles, and multilamellar vesicles. Liposomes can be formed from a variety of lipids, such as cholesterol, stearylamine, or phosphatidylcholines. A compound of the invention may also be delivered using antibodies, antibody fragments, growth factors, hormones, or other targeting moieties to which the compound molecules are coupled (e.g., see Remington: The Science and Practice of Pharmacy, vide supra), including in vivo conjugation to blood components of a compound of the formula I or II, as described herein.

### In Vitro Application in Identification of TLR Antagonists:

Pharmaceutical agents that can be used in the therapeutic methods of the invention can be identified in screening methods. For example, cell-based screening methods can be used, in which cells expressing TLR are contacted with a candidate agent and the impact of the agent on the activation of TLR in the cells is determined. In one example of such a method, the effect of an agent on the activation of TLR by a known ligand (e.g., a lipopeptide,) is determined. Agents that are found to decrease or to block activation of the receptor by the ligand can then be considered for further analysis and/or for use as TLR inhibitors in therapeutic methods. Activation of TLR in these methods can be measured using, for example, a reporter system. For example, cells used in the screening assay can include a reporter gene that is under the control of a promoter that is inducible by a signaling pathway triggered by TLR activation.

In addition to cell-based methods, candidate agents can be tested in animal model systems. This may be desirable, for example, if an agent has been found to have antagonist activity in a cell-based assay or to bind to TLR in an in vitro assay (see below). For example, in animal studies, test agents can be administered to an animal model concurrently with a molecule known to activate TLR (e.g., lipopeptide), and the impact of the agent on a response in the animal that is normally triggered by activation of the receptor (e.g., cytokine induction) can be determined. Further, in vitro methods can be used. For example, a candidate compound can be assayed for whether it binds to TLR or a fragment of the receptor that includes at least a portion of the ligand binding site. Such assays can be carried out using, for example, columns or beads to which the receptor or fragment is bound.

In addition to the methods described above, additional TLR antagonists can be identified in methods in which candidate compounds are compared for TLR antagonist activity with any of the TLR antagonists described herein. Further, in addition to being compared for TLR antagonist activity, the candidate compounds can be compared with TLR2 antagonists with respect to specificity for TLR versus other receptors. Candidate compounds identified as having TLR antagonist activity that is, for example, similar to or greater than the activity of the antagonists described herein (and/or with similar or greater levels of specificity for TLR2 versus TLR4) in these assays can be tested further, for example, in appropriate animal model assays for any of the diseases or conditions described herein, as well as in human clinical studies.

Also included in the invention are compounds that are selective for TLR2 over TLR4, as well as compounds that are dual antagonists (i.e., antagonists of both TLR2 and TLR4). A compound that is selective for TLR2 over TLR4 is one that has, for example, an IC₅₀ value in a TLR2 antagonist assay, such as is described herein, that is less than that found in a TLR4 antagonist assay. For example, the IC₅₀ in the TLR2 assay can be at least 5, 10, 25, or 50-fold less than the value for the same compound tested in the TLR4 assay. Compounds that are dual antagonists are those that have, for example, IC₅₀ values that are within a 5-fold range of one another using. Thus, dual antagonists include those that have activities that are 1:5 5:1 with respect to one another (e.g., 1:4, 1:3, 1:2, 1:1, 2:1, 3:1, and 4:1). The invention also includes the use of TLR1 antagonists such as those described herein in the study of physiological and molecular pathways involved in or affected by TLR2 activation (or inactivation).

Agents that can be screened using the methods of the invention include, for example, compounds that are present in compound libraries (e.g., random libraries), as well as analogs of known TLR2 ligands (e.g., lipopeptides) that are modified to prevent rather than activate TLR2. Further, peptides that correspond to the binding site of TLR2 or its ligands, which can competitively inhibit ligand binding, can be tested. Further, antibodies or antibody fragments to the ligand or the ligand binding site of the receptor can be screened.

The following examples are included to demonstrate embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The invention will now be illustrated with the aid of following non-limiting examples. It should be understood, however, that the invention is not limited to the solely to the particular examples given below. It will be apparent that those skill in the art that any modifications, both to the materials and methods, may be practiced without departing from the purpose and interest of this invention

a) All operations which were carried out at room temperature or ambient temperature were in the range of 18 to 25° C.

b) Evaporation of the solvent was carried out under reduced pressure (600-4000 pascals; 4.5-30 mm Hg) with a bath temperature up to 40° C.

c) The course of the reaction was monitored by thin layer chromatography (TLC) and reaction times are given for illustration only.

d) Melting points are uncorrected, the melting points are given for the materials prepared as described, and polymorphism may result in isolation of materials with different melting points in some preparations.

e) The structure and purity of all final products were assured by at least one of the following techniques: TLC, NMR (nuclear magnetic resonance) spectroscopy, IR (Infrared spectroscopy), or microanalytical data and HPLC

f) Yields are given for illustration only.

g) When given, NMR data is in the form of delta (A) values for major diagnostic protons given in parts per million (ppm) relative to tetramethylsilane (TMS) as internal standard determined at 300 MHz or 400 MHz using the indicated solvent.

h) Chemical symbols have their usual meanings; the following abbreviations have also been used: v (volume), w (weight), B.P. (boiling point), M.R. (Melting range), M.pt. (melting point), L (liters), ml (milliliters), gms (grams), mg (milligrams), mol (moles), mmol (millimoles) eq (equivalents) deg C (degree centigrade), conc. HCl (concentrated hydrochloric acid) any other.

### General Methods for Preparation of TLR Antagonists

### Example-1

### Isolation of 1,5-dihydroxy-2,7-dimethoxy-9,10-dihydrophenanthrene (Eulophiol) (RSCL-520) and 2,7-dihydroxy-3,4-dimethoxyphenanthrene (Nudol) (RSCL-518)

### 1. Collection of Plant Materials:

Tubers of Eulophia ochreata were collected in the month of October 2006. Herbarium voucher specimen (Accession No. 157) was authenticated and deposited at herbarium of Dhirubhai Ambani Life Sciences Center, Navi Mumbai, Maharashtra.

### 2. Extraction of Plant Materials: (Scheme-I)

Tubers were chopped in small pieces, shade dried and pulverized. Powder (1 kg) was extracted under stirring with 6ltr of pet ether (60-80) at room temperature for 7 hr. The reaction mixture was filtered by using buchner funnel. Organic filtrate was stored and the cake was reextracted with 6ltr of pet ether (60-80) under above conditions. The extraction was repeated one more time. The three extracts of pet ether were combined (about 15ltr) and concentrated under vacuum to get dark yellow residue (4.6 g). The cake was further extracted with dichloromethane three times using 6ltr of solvent each time. The three extracts of dichloromethane (about 15ltr) were mixed and concentrated under vacuum to yield dark brown residue (24 g). Cake obtained after dichloromethane extraction was further extracted three times with ethyl acetate and followed by methanol using 6ltr solvent each time. The removal of ethyl acetate (about 16ltr) under vacuum gave dark black solid (6.4 g) and concentration of methanol extract (about 17ltr) under vacuum gave dark brown lump (4.9 g).

### 3. Testing of Free Radical Scavenging Activity of Various Fractions:

Residue obtained after removal of solvents were tested for free radical scavenger activity using curcumin as reference standard and DPPH (2,2-Diphenyl-1-picryl-hydrazyl) as radical.

Test solution preparation: DPPH was dissolved in methanol (AR grade) to a concentration of 10 mM.

Sample preparation: Weighed about one mg of extract residue/curcumin and dissolved in 100 μl of DMSO and added methanol in order to make concentration 1 mg/ml. Pipetted out 50 μl sample solution in a 96-well micro titre plate to which added 200 μl of above prepared DPPH test solution. The plate was incubated in dark for half an hour. Absorbance measured at 540 nm on Eliza plate reader. The corresponding blank readings were also taken for calculating the percentage antioxidant activity. Antioxidant activity in percentage was calculated by the formula: (1−(Absorbance of sample/Absorbance of DPPH)×100.

TABLE 1
Free radical scavenging activity of
different extracts in DPPH assay.

| Extract residue/Compound | Antioxidant activity (% age) |
|---|---|
| Curcumin* | 92.02 |
| Pet ether extract residue | 44.96 |
| Dichloro methane extract residue | 93.7 |
| Ethyl acetate extract residue | 93.7 |
| Methanol extract residue | 48.53 |

*Reference standard.

4. Isolation of EULOPHIOL and NUDOL from Dichloromethane Fraction:

Based on free radical scavenging activity of different fractions as above, the residue of both dichloromethane and ethyl acetate extracts were combined and chromatographed over 300 g silica gel (60-120 mesh) in a glass column (90×5 cm). A mixture of pet ether and ethyl acetate in different combinations were used as mobile phase. The eluted fractions with pet ether and ethyl acetate (89:11 v/v) containing Eulophiol were collected where as the fractions eluted with pet ether and ethyl acetate (89:15 v/v) were collected for nudol. TLC monitoring system was pet ether and ethyl acetate (45:55, v/v) as the mobile phase. RF values calculated for eulophiol and nudol were 0.7 and 0.6 respectively.

### 5. Purification and Characterization:

### Eulophiol:

All fractions containing Eulophiol were mixed and concentrated to remove the total solvent under vacuum. The syrupy mass left at room temperature for a week to get white crystals. The crystals were suspended in dichloro methane (5 ml), filtered, washed and dried in oven under vacuum to get Eulophiol. Yield: 0.150 g (0.014%) (Reported⁹ 0.21%). M.P. 201-203° C. (lit⁹., 202-203); ¹H NMR: (400 MHz, CD₃COCD₃, δ ppm) δ 2.59-2.75 (m, 4H, H-9 and H-10), δ 3.82 (S, 3H, OMe), δ 3.85 (S, 3H, OMe), δ 6.39 (d, 1H, J=2.4 Hz, meta coupled, H-6), δ 6.45 (d, 1H, J=2.4 Hz, meta coupled, H-8), δ 6.78 (d, 1H, J=8.4 Hz, ortho coupled, H-3), δ 7.28 (br s, 1H, exch.D₂O, OH), δ 7.72 (d, 1H, J=8.4 Hz, ortho coupled, H-4), δ 8.37 (br s, 1H, exch.D₂O, OH); MS: m/z 273 (M+1)

HPLC analysis showed 98.89% purity. Analysis was performed on column Hypersil GOLD C18, 5 μm, 4.6×250 mm, Wave length: 265 nm, Mobile phase: Buffer: Acetonitrile, Buffer: 2 ml TEA=1 litre water. Adjust pH to 3.0 with phosphoric acid, Run time: 60 min, Injection volume: 20 μl, Sample preparation: 0.1 mg/ml, Diluent: Acetonitrile.

### Structure of Eulophiol (RSCL-0520):

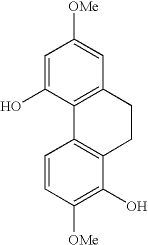

### Nudol:

All the fractions containing Nudol were mixed and concentrated to minimum volume of 3-4 ml and added 10-12 ml of pet ether to form the precipitate. Precipitate was stirred in pet ether, filtered and washed. Dark brownish hard crystals of nudol got separated on filter paper. Yield: 0.663 g (0.063%). (Reported yield is 0.003%; Bhandari S R, Kapadi A H, Majumder P L, Joardar, Mukta, Shoolery J N. Nudol, a phenanthrene of the orchids Eulophia nuda, Eria carinata and Eria stricta. Phytochemistry. 1985; 24: 801-804.). M.P: 253° C. (lit., 253; Id.); ¹H NMR: (400 MHz, CDCl₃, δ ppm) δ 3.95 (S, 3H, OMe), δ 4.05 (s, 3H, OMe), δ 5.08 (br s, 1H, exch.D₂O, OH), δ 6.01 (br s, 1H, exch.D₂O, OH), δ 7.08 (s, 1H, H-1), δ 7.18 (dd, 1H, J=9.2 Hz, J=2.8 Hz, ortho and meta coupled, H-6), δ 7.22 (d, 1H, J=2.8 Hz, meta coupled, H-8), δ 7.53 (AB-quartet, 2H, J=9.0 Hz, H-9 and H-10), δ 9.33 (d, 1H, J=9.2 Hz, ortho coupled, H-5); MS: m/z 271 (M+1).

HPLC analysis showed 97.02% purity. Analysis was performed on column Hypersil GOLD C18, 5 μm, 4.6×250 mm, Flow rate: 1 ml/min, Wave length: 257, 275 nm, Mobile phase: Buffer: Acetonitrile, Buffer: 2 ml TEA=1 litre water. Adjust pH to 3.0 with phosphoric acid, Run time: 35 min, Injection volume: 20 μl, Sample preparation: 0.1 mg/ml, Diluent: Acetonitrile.

### Structure of Nudol (RSCL-0518)

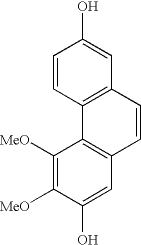

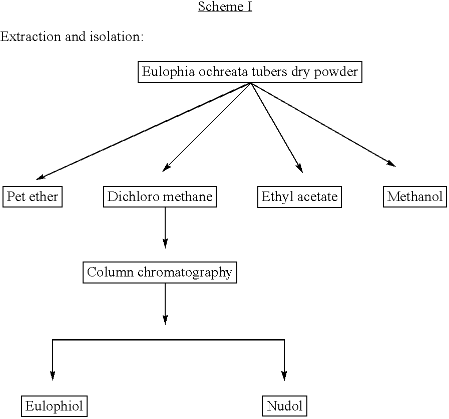

### Example 2

### Derivatization of Isolated Molecules

### A. Synthesis of 1,5-diacetoxy-2,7-dimethoxy-9,10-dihydrophenanthrene¹ (RSCL-0521)

To a solution of eulophiol (I) (0.020 g, 0.07 mmol) in dichloromethane (20 ml) was added acetic anhydride (0.020 ml), pyridine (0.020 ml) and stirred overnight at R.T. (20-25° C.). To this was then added water (50 ml) and the organic layer washed with dilute hydrochloric acid (10 ml), water (10 ml), dried over sodium sulfate and concentrated under reduced pressure, which gave pure compound (III) (RSCL-0521) (0.022 g) in 84.61% yield (w/w). M.P. (143-144° C. (reported by Bhandari S R et al. Phytochemistry. 1983; 22: 747-48), 143° C.; ¹H NMR: (400 MHz, CD₃COCD₃, δ ppm) δ 2.25 (S, 3H, OAc), δ 2.29 (S, 3H, OAc), δ 2.59-2.70 (m, 4H, H-9 and H-10), δ 3.84 (S, 3H, OMe), δ 3.89 (S, 3H, OMe), δ 6.66 (d, 1H, J=2.4 Hz, meta coupled, H-6), δ 6.78 (d, 1H, J=2.4 Hz, meta coupled, H-8), δ 6.96 (d, 1H, J=8.4 Hz, ortho coupled, H-3), δ 8.16 (d, 1H, J=8.4 Hz, ortho coupled, H-4), MS: m/z 357 (M+1).

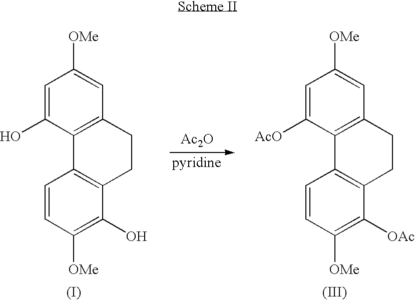

### B. Synthesis of 2,7-diacetoxy-3,4-dimethoxyphenanthrene² (RSCL-0519)

To a solution of nudol (II) (0.020 g, 0.07 mmol) in dichloromethane (20 ml) was added acetic anhydride (0.020 ml), pyridine (0.020 ml) and stirred overnight at R.T. (20-25° C.). To this was then added water (50 ml) and the organic layer washed with dilute hydrochloric acid (10 ml), water (10 ml), dried over sodium sulfate and concentrated under reduced pressure, which gave pure compound (IV) (RSCL-0519) (0.108 g) in 82.44% yield (w/w). M.P. 151° C., (151° C.; Bhandari et al. Phytochemistry. 1985; 24: 801-804.); ¹H NMR: (400 MHz, CDCl₃, δ ppm) δ 2.37 (s, 3H, OAc), δ 2.46 (s, 3H, OAc), δ 3.92 (s, 3H, OMe), δ 3.98 (s, 3H, OMe), δ 7.15 (s, 1H, H-1), δ 7.36 (dd, 10H, J=9.2 Hz, J=2.8 Hz, ortho and meta coupled, H-6), δ 7.58 (d, 1H, J=2.8 Hz, meta coupled, H-8), δ 7.64 (apparent singlet (AB-quartet), 2H, H-9 and H-10), δ 9.42 (d, 1H, J=8.8 Hz, ortho coupled, H-5) MS: m/z 355 (M+1).

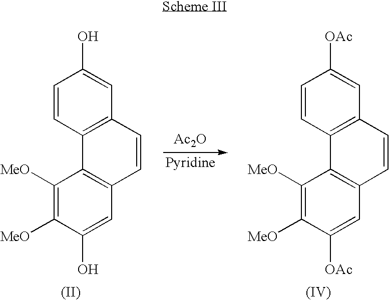

### C. Synthesis of 2,3,4,7-tetramethoxyphenanthrene² (RSCL-0575)

To a solution of nudol (II) (0.027 g, 0.1 mol) in acetone (20 ml) was added Potassium Carbonate (0.069 g, 0.5 mmol), Methyl Iodide (0.5 ml) and stirred at RT (20-25° C.) overnight. The reaction mixture was then filtered and the compound (V) (RSCL-0575) was isolated by silica gel column chromatography (Pet-ether-Ethyl acetate gradient). Yield=0.0236 g (79.19% w/w) M.P. 148, (148° C.; Bhandari et al. Phytochemistry. 1985; 24: 801-804.); ¹H NMR: (400 MHz, CDCl₃, 6 ppm) δ 3.95 (s, 3H, OMe), δ 4.00 (s, 3H, OMe), δ 4.03 (s, 3H, OMe), δ 4.05 (s, 3H, OMe), δ 7.00 (s, 1H, H-1), δ 7.16 (dd, J=9.2 Hz, J=2.8 Hz, ortho and meta coupled, H-6), δ 7.19 (d, 1H, J=2.8 Hz, meta coupled, H-8), δ 7.51 (apparent singlet (AB-quartet), 2H, H-9 and H-10), δ 9.34 (d, 1H, J=9.2 Hz, ortho coupled, H-5), MS: m/z 299 (M+1).

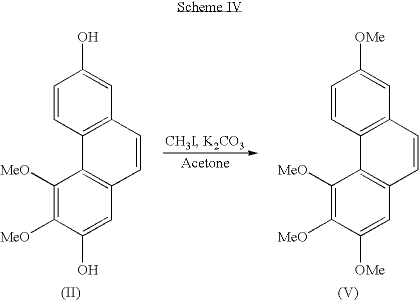

### Example 3

### Synthesis of Novel TLR Antagonists

### 1. Synthesis of 3,4-dimethoxyphenanthrene-2,7-bis-[(2E)-3-[3,4-bis(acetyloxy)phenyl] acrylate (IX) (RSCL-0522)

### A. Synthesis of 3,4-Di-O-acetylcaffeic acid (VII)

To a solution of 3,4-Dihydroxycinnamic acid (VI) (1.8 g, 10 mmol) in anhydrous pyridine (1.8 ml) was added acetic anhydride (2.25 g, 22 mmol) and stirred overnight at room temperature (20-25° C.). The reaction was quenched by adding water (5 ml) and extracted with ethyl acetate (3×50 ml). The ethyl acetate layer washed successively with saturated sodium bicarbonate, brine and dried over anhydrous sodium sulfate. After concentration under reduced pressure the product (VII) was recrystallized from methanol.

Yield 2.2 g (83%), M.P 198-199° C., ¹H NMR: (400 MHz, CDCl₃, δ ppm) 2.32 (s, 6H, two OAc), 6.40 (d, 1H, Hα, J_{αβ}=16.0 Hz), 7.25 (d, 1H, H-5, J_{5,6}=8.8 Hz), 7.39 (d, 1H, H-2, J_{2,6}=2.0 Hz), 7.44 (dd, 1H, H-6, J_{6,5}=8.8 Hz, J_{6,2}=2.0 Hz), 7.72 (d, 1H, Hβ, J_{βα}=16.0 Hz,) MS: m/z 287 (M+Na)

### B. Synthesis of 3,4-Di-O-acetylcaffeoyl chloride (VIII)

To a solution of 3,4-di-O-acetylcaffeic acid (VII) (0.063 g, 0.24 mmol) in anhydrous ethylene dichloride (10 ml) were added thionyl chloride (1 ml), N,N′-dimethyl formamide (Cat) and refluxed in an oil bath (80-90° C.) for 1 hr. The excess solvent and reagent were distilled off & pure acyl chloride (VIII) was immediately used as such without further purification.

### C. Synthesis of 3,4-dimethoxyphenanthrene-2,7-bis-[(2E)-3-[3,4-bis(acetyloxy)phenyl] acrylate (IX)

To the above acyl chloride (VIII) in dichloromethane (50 ml) was added nudol (II) (0.027 g, 0.1 mmol) and stirred overnight at room temperature (20-25° C.). The reaction was quenched by adding water (10 ml) and extracted with dichloromethane (3×50 ml). The dichloromethane layer was washed successively with saturated sodium bicarbonate, brine and dried over anhydrous sodium sulfate. After concentration under reduced pressure the product (IX) (RSCL-0522) was isolated by silica gel column chromatography (Pet-ether-ethyl acetate gradient). Yield 0.036 g (47%), M.P. 174-175° C., ¹H NMR: (400 MHz, CDCl₃, δ ppm) δ 2.33 (S, 12H, OAc), δ 3.95 (S, 3H, OMe), δ 3.99 (S, 3H, OMe), δ 6.65 (d, 1H, J=16 Hz), δ 6.76 (d, 1H, J=16 Hz), δ 7.19 (S, 1H), δ 7.28 (d, 1H), δ 7.30 (d, 1H), δ 7.40-7.55 (m, 5H), δ 7.68 (d, 3H), δ 7.87 (d, 1H, J=16 Hz), δ 7.93 (d, 1H, J=16.4 Hz), δ 9.47 (d, 1H, J=9.2 Hz). MS: m/z 785 (M+Na)

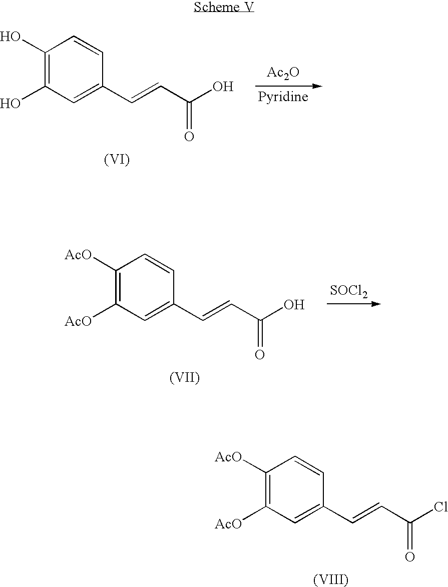

### 2. Synthesis of 1,5-dibenzyloxy-2,7-dimethoxy phenanthrene (RSCL-0638)

### A. Synthesis of the intermediates 6,6′-dibenzyloxy-4,4′-dimethoxy-biphenyl-2,2′-dicarbaldehyde (XII), 3,6′-dibenzyloxy-4,4′-dimethoxy-biphenyl-2,2′-dicarbaldehyde) (XIII), (3,3′-dibenzyloxy-4,4′-dimethoxy-biphenyl-2,2′-dicarbaldehyde) (XIV)

Synthesis of the intermediates 6,6′-dibenzyloxy-4,4′-dimethoxy-biphenyl-2,2′-dicarbaldehyde(XII), 3,6′-dibenzyloxy-4,4′-dimethoxy-biphenyl-2,2′-dicarbaldehyde) (XIII), (3,3′-dibenzyloxy-4,4′-dimethoxy-biphenyl-2,2′-dicarbaldehyde) (XIV were first carried out as per the procedure reported in the literature (Spencer B. Jones, Liwen He, and Steven L. Castle, Total Synthesis of (±)-Hasubanoine, Org. lett. 2006; 8: 3757-3760.).

A three necked flask previously flushed with argon was charged with bis(pinacolato diboron) (3.61 g, 14.21 mmol), Potassium acetate (3.60 g, 36.71 mmol), PdCl₂(dppf), CH₂Cl₂ (0.29 g, 0.35 mmol), dimethyl sulphoxide (35 ml), stirred for 5-10 min, added a solution of 3-Benzyloxy-2-bromo-5-methoxybenzaldehyde (X) (3.8 g, 11.84 mmol) in dimethyl sulphoxide (15 ml) slowly over a period of 10-15 min. (Mark A. Rizzacasa and Melvyn V. Sargent, Synthetic Approaches to the Alkaloids of the Ancistrocladaceae. Part 3. The Total Synthesis of (−)-Ancistrocladinine: Control of the Diastereoisomer Excess in the Synthesis of Axially Chiral Biaryls, J Chem. Soc. Perkin. Trans. I. 1991; 2773-2781) The resulting mixture was heated gently for two hours at 80° C. Cooled to room temperature (20-25° C.), added Potassium carbonate (5.24 g, 37.9 mmol), PdCl₂(dppf) CH₂Cl₂ (1.16 g, 1.42 mmol), a solution of 2-Benzyloxy-6-bromo-3-methoxybenzaldehyde (IV) (4.55 g, 14.17 mmol) in 100 ml dimethyl sulphoxide (90 ml+10 ml rinse) and heated gently for two hours at 80° C. (Takeshi Nakanishi, Masanobu Suzuki, Akihiro Mashiba, Keizou Ishikawa and Takashi Yokotsuka, Synthesis of NK109, an anticancer Benzo(c)phenanthridine Alkaloid, J. Org. Chem. 1998; 63: 4235-4239) The reaction quenched by adding water (200 ml), added excess sodium chloride & extracted with ethyl acetate (3×50 ml), washed the ethyl acetate layer with brine (50 ml), dried over sodium sulfate, and the mixture was subjected to silica gel column chromatography (pet ether-ethyl acetate gradient). This gave compound XII (0.9 g), compound XIII (2.0 g) and compound XIV (0.6 g) as semi solid materials which solidified on standing for 24 hr at R.T (20-25° C.).

Compound XII (6,6′-dibenzyloxy-4,4′-dimethoxy-biphenyl-2,2′-dicarbaldehyde) M.P. 90-95° C., ¹HNMR: (400 MHz, CDCl₃, δ ppm) δ 3.81 (s, 6H, two OCH₃), 4.91 (m, 4H, CH₂), 6.73 (d, 2H, J=2.4 Hz, ArH), 7.0-7.2 (m, 12H), 9.64 (s, 2H, CHO), MS: m/z 483 (M+1).

Compound XIII (3,6′-dibenzyloxy-4,4′-dimethoxy-biphenyl-2,2′-dicarbaldehyde). M.P. 78-85° C., ¹HNMR: (400 MHz, CDCl₃, δ ppm) δ 3.79 (s, 3H, OCH₃), 3.79 (s, 3H, OCH₃), 3.90 (s, 3H, OCH₃), 4.86 (s, 2H, CH₂), 5.1 (m, 2H, CH₂), 6.62-7.40 (m, 14H, ArH), 9.55 (s, 1H, CHO), 10.10 (s, 1H, CHO), MS: m/z 483 (M+1)

Compound XIV (3,3′-dibenzyloxy-4,4′-dimethoxy-biphenyl-2,2′-dicarbaldehyde). M.P. 68-70° C., ¹HNMR: (400 MHz, CDCl₃, δ ppm) δ 3.87 (s, 6H, two OCH₃) 5.10 (m, 2H, CH₂), 5.1 (m, 2H, CH₂), 6.75 (d, 2H, J=8.4 ArH), 7.20-7.40 (m, 10H, ArH), 10.03 (s, 2H, CHO), MS: m/z 483 (M+1)

### B. Synthesis of 6,6′-dibenzyloxy-4,4′-dimethoxy-2,2′-divinyl-biphenyl (XV)

To a suspension of methyltriphenylphosphonium bromide (0.37 g, 1.037 mmol) in anhydrous tetrahydrofuran (11.6 ml) was added dropwise n-Butyl Lithium (0.74 mLl, 1.6 M in hexanes, 1.18 mmol) at 0° C. The solution was warmed to RT (20-25° C.) and stirred for three hours, then cooled to −78° C. and added a solution of 6,6′-dibenzyloxy-4,4′-dimethoxy-biphenyl-2,2′-dicarbaldehyde (XII) (0.1 g, 0.207 mmol) in anhydrous tetrahydrofuran (10.4 ml) dropwise over a period of ten minutes. The resultant mixture was stirred for ten minutes, then warmed to RT (20-25° C.) and stirred overnight. The reaction was quenched by the addition of 1N hydrochloric acid (pH changed to 4-5), diluted with brine (20 ml), extracted with ethyl acetate (3×20 ml), dried over sodium sulfate, and the product, an oil, was isolated by silica gel column chromatography (pet-ether-ethyl acetate gradient). Yield=0.058 g (58.58%), ¹HNMR: (400 MHz, CDCl₃, δ ppm) δ 3.77 (s, 6H, two OCH₃), 4.88 (s, 4H, CH₂), 5.00 (d, 2H, J=10.8), 5.54 (d, 2H, J=18.0 Hz), 6.30 (m, 2H), 6.41 (d, 2H, J=2.4 Hz), 6.76 (d, 2H, J=2.4 Hz), 7.0-7.2 (m, 10H) MS: m/z 479 (M+1)

### C. Synthesis of 1,5-dibenzyloxy-2,7-dimethoxy phenanthrene (XVI)

To a solution of 6,6′-dibenzyloxy-4,4′-dimethoxy-2,2′-divinyl-biphenyl (XV) (0.048 g, 0.1 mmol) in dichloromethane (10 ml) was added the Grubbs second-generation ruthenium catalyst (0.0025 g, 0.003 mmol), refluxed for two hours under argon, concentrated the reaction mixture in vacuo and the product was isolated by silica gel column chromatography (pet-ether-ethyl acetate gradient). Yield=0.020 g (44%) M.P. 75-80° C., ¹HNMR: (400 MHz, CDCl₃, δ ppm) δ 3.86 (s, 6H, two OCH₃), 4.80 (s, 4H, CH₂), 6.62 (d, 2H, J=2.4, ArH), 6.77 (d, 2H, J=2.4 Hz, ArH), 7.0-7.2 (m, 10H, ArH), 7.45 (s, 2H, H-9 & H-10), MS: m/z 451 (M+1)

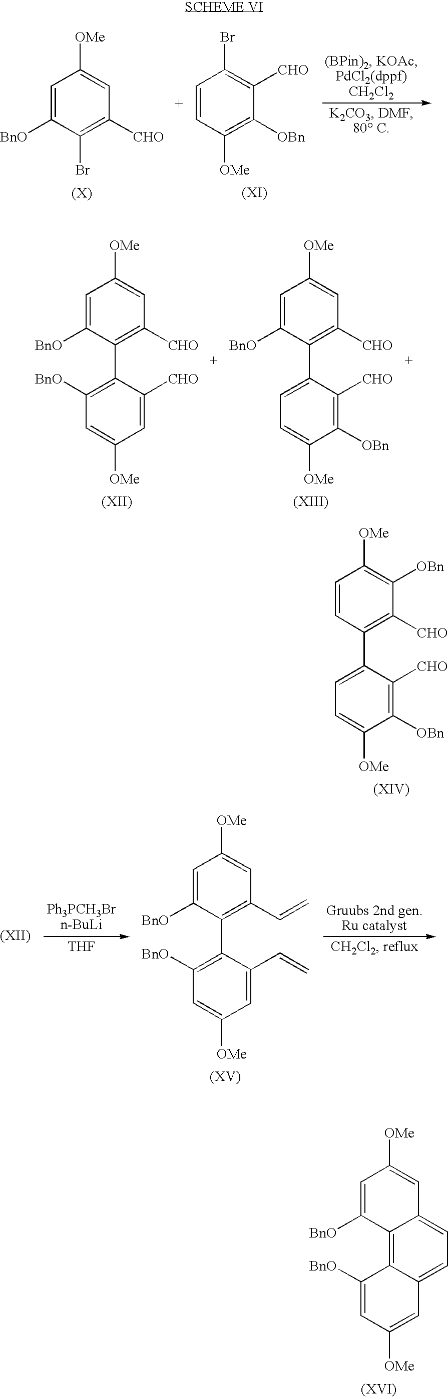

### Example 9

### In-Vitro Testing

### Experiment 1: Comparative TNF Inhibition Activity Chart of Isolated and Synthesized Molecules:

The molecules isolated and synthesized were initially pre-screened for TNF inhibition activity to identify the most effective anti-inflammatory molecule. (Table 2). RSCL-0520 showed the best TNF inhibiting activity, which was investigated for further in-vitro studies.

TABLE 2
Comparative analysis of anti-TNF activity
of isolated/synthesized molecules.

| | % TNF Inhibition | | | | |
|---|---|---|---|---|---|
| | Cells + | Cells + | Cells + | Cells + | |
| | 100 μM + | 50 μM + | 10 μM + | 1 μM + | Cells + |
| | LPS | LPS | LPS | LPS | LPS |
| RSCL-0518 | 87 | 39 | 0 | 0 | 0 |
| RSCL-0519 | 72 | 21 | 0 | 0 | 0 |
| RSCL-0520 | 100 | 95 | 40 | 4 | 0 |
| RSCL-0521 | 92 | 76 | 13 | 0 | 0 |
| RSCL-0522 | 34 | 37 | 12 | 0 | 0 |
| RSCL-0575 | 49 | 32 | 33 | 26 | 0 |
| RSCL-0638 | 0 | 0 | 0 | 0 | 0 |

### Experiment 2: Effect of RSCL-0520 on Various TLR Ligands

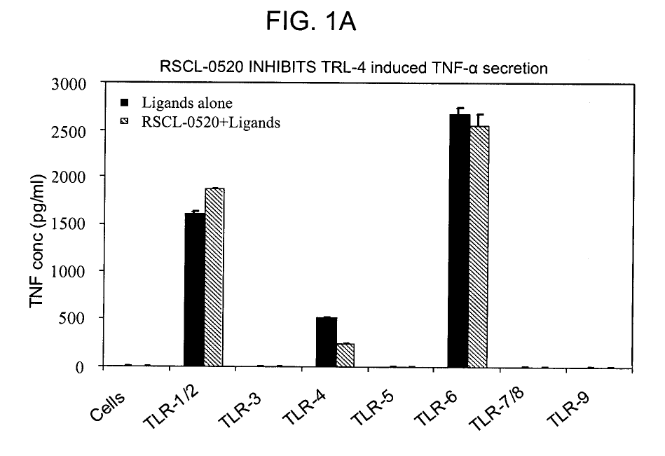

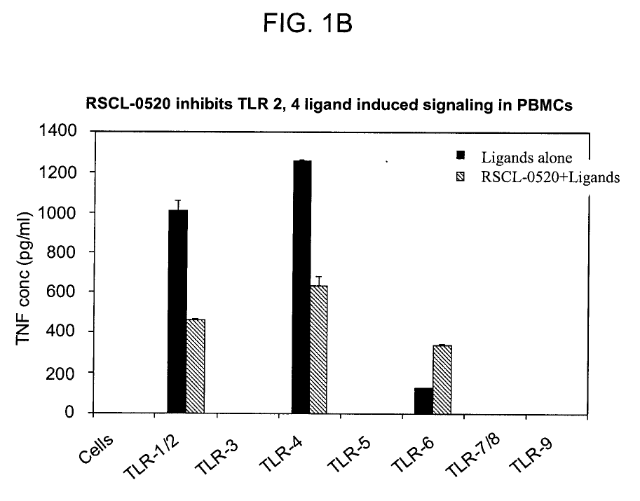

The present invention has checked the effect of various TLR ligands on THP-1 monocytes, PBMCs and their ability to activate and release TNF-α. For this purpose about 9 TLR ligands were used. These ligands were obtained from Apotech Cat; APO-54N-018-K101 and assayed for TNF-α release in culture supernatants. THP-1 (FIG. 1a) and PBMCs (2×10⁵ cells/well) (FIG. 1b) were plated in 96-well plate. The cells were pretreated with RSCL-0520 in DMSO 1 hr prior to TLR ligand treatment. Following 1 hr pre-treatment, the cells were treated with TLR ligands [TLR2, TLR5 and TLR6-75 ng/ml each, TLR3-75 μg/ml, TLR4-750 ng/ml, TLR7, TLR8 and TLR9-7.5 μg/ml) for 24 hrs. The culture supernatant were collected after the stipulated time and assayed for TNF-α release using a Duoset Enzyme-Linked ImmunoSorbent Assay (ELISA) detection Kit (R&D systems, MN 55413, USA; Cat: DY-210 E). Simultaneously, supernatants from cells treated with ligands without RSCL-0520 pre-treatment and RSCL-0520 without ligand treatment were collected which served as respective controls.

We detected TNF-α secretion from cells stimulated with TLR2, TLR4 and TLR6. No detectable TNF-α was observed following stimulations with other ligands. In THP-1 cells pretreated with RSCL-0520, TNF-α production decreased by ˜50% following TLR4 ligand stimulation. Further, the same inhibitory effect was seen in PBMCs (˜%50 TLR4 following TLR4 ligand stimulation. These results show that RSCL-0520 selectively inhibits TNF-α production mediated mainly by TLR4 in THP-1 and its inhibition in addition to TLR4 extended to TLR2 in PBMCs. This clearly indicates that RSCL-0520 exerts its inhibition primarily TLR-4 signaling mechanisms.

### Experiment 3: Inhibition of TNF-α Secretion in THP-1 Cells by RSCL-0520 is Dose-Dependent

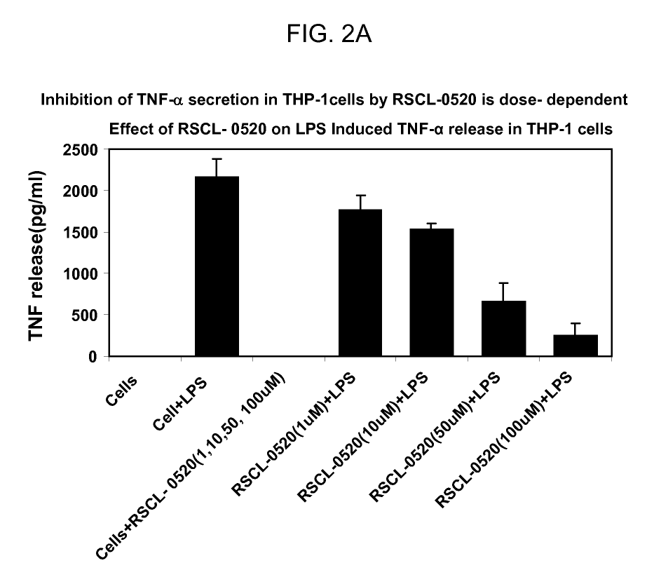

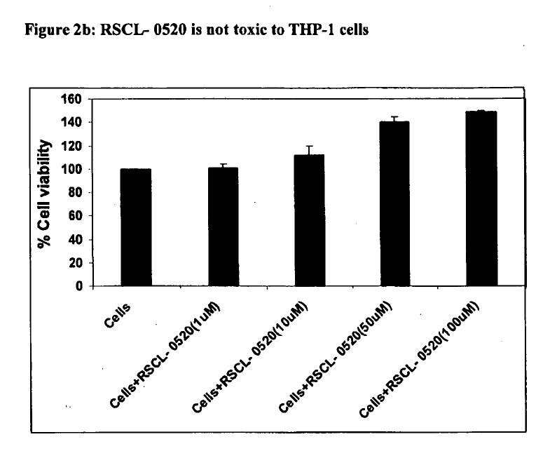

To confirm whether the inhibitory effect of RSCL-0520 is dose-dependent, the present invention has studied the ability of RSCL-0520 to inhibit TNF-α secretion from LPS (250 ng/ml) induced THP-1 monocytes (FIG. 2a).

THP-1, 2×10⁵ cells/well was plated in 96-well plate. The cells were pretreated with RSCL-0520 at various concentrations (100 μM, 50 μM, 10 μM and 1 μM) 1 hr prior to LPS stimulation. As a control group, cells were treated with LPS alone and cells treated with RSCL-0520 alone were used. TNF-α secretion was estimated in the culture supernatants following 24 hr LPS stimulation using Duoset ELISA detection Kit (R&D systems, MN, USA). ***P<0.001 values are comparisons for LPS treated vs. RSCL-0520 treated, NS indicates not significant.

The toxicity of RSCL-0520 (FIG. 2b) was also by treating cells with RSCL-0520 by MTT assay. LPS induced TNF-α secretion was inhibited by RSCL-0520 in a concentration dependent manner. The viability of the cells (analyzed in tandem by MTT) was not affected by RSCL-0520 indicating its non-toxic nature.

### Experiment 4: Effect of RSCL-0520 on LPS Induced TNF-Release in Human PBMC

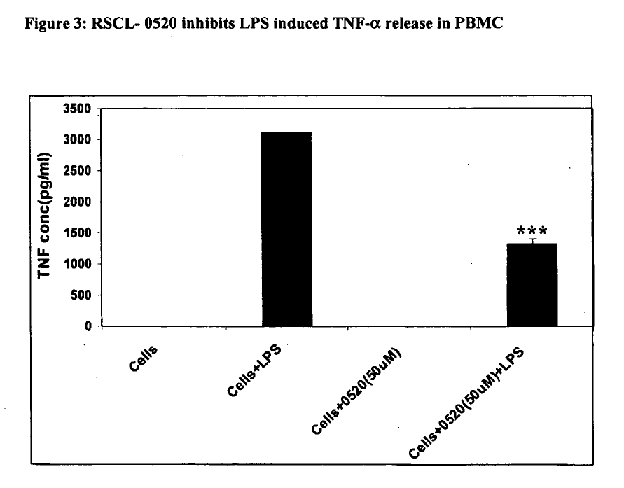

To evaluate the ability of RSCL-0520 to inhibit LPS induced TNF-α secretion in a physiological scenario, the present invention has tested the same in PBMC isolated from human blood (FIG. 3) by standard methodology. The inhibition was similar to that observed in THP-1 cells. The TNF levels were not detectable in PBMCs without LPS and with RSCL-0520 treatment alone, indicating its specificity in LPS induced TNF-α through a TLR mediated process. ***P<0.001 value is for LPS treated vs. RSCL-0520 treated.

### Experiment 5: Effect of RSCL-0520 on TNF Response to Increasing Concentration of LPS

The present invention has also conducted experiments to check the ability of RSCL-0520 to inhibit LPS induced TNF-α secretion from THP-1 cells, wherein the studies involved the stimulation of THP-α cells with increasing concentrations of LPS (31.25 ng/ml to 1000 ng/ml) with and without pre-treatment of cells with different concentrations of RSCL-0520.

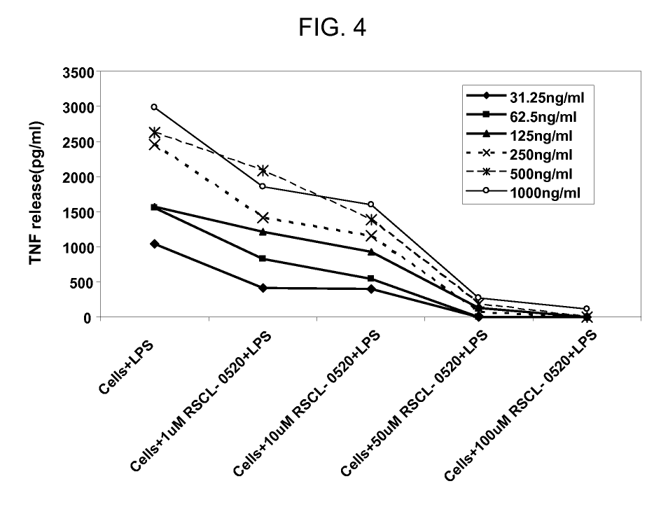

It is clearly evident (FIG. 4) that with an increased secretion of TNF-α with increasing concentration of LPS is inhibited by RSCL-0520 to varied extents on a concentration dependent manner. Further, even RSCL-0520 (10 μM) was effective to inhibit LPS (1000 ng/ml) induced TNF-α secretion; clearly indicating it's potency as an antagonist to LPS induced processes.

Experiment 6: Effect of RSCL-0520 on TNF-α mRNA Expression in THP-1 Cells

To determine whether the suppressive effect of RSCL-0520 on the cytokine production occurs at mRNA expression level, quantitative real-time PCR was used to examine TNF-α mRNA expression in THP-1 cells stimulated with LPS. THP-1 cells (3×10⁶ cells/well) were stimulated with 250 ng/ml LPS in the presence or absence of RSCL-0520 (50 μM) for the 1 hr. Total RNA was isolated from these cells and cDNA was synthesized. LPS treated cells acted as positive control. All quantitative real-time PCR (TaqMan™) primers and probes were obtained from Applied Biosystems (ABI), Weiterstadt, Germany. For detection of TNF-α, pre-developed assay reagents (Universal master mix as obtained from Applied Biosystems included all reagents including Taq-polymerase apart from specific primers and probes) were used. The PCR was performed utilizing 1 μl cDNA per reaction in triplicates of 25 μl volume on an ABI 7500 Realtime PCR machine using a 2-step PCR protocol.

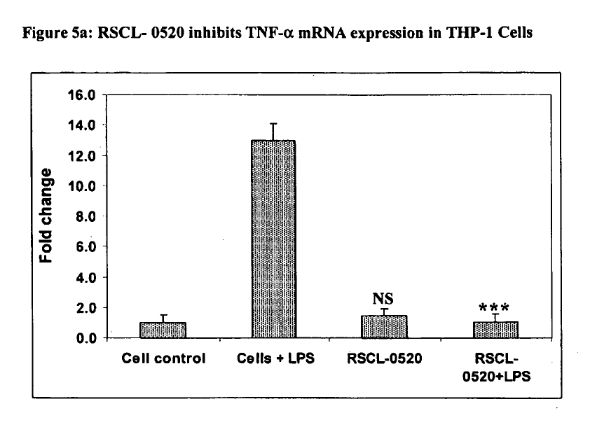

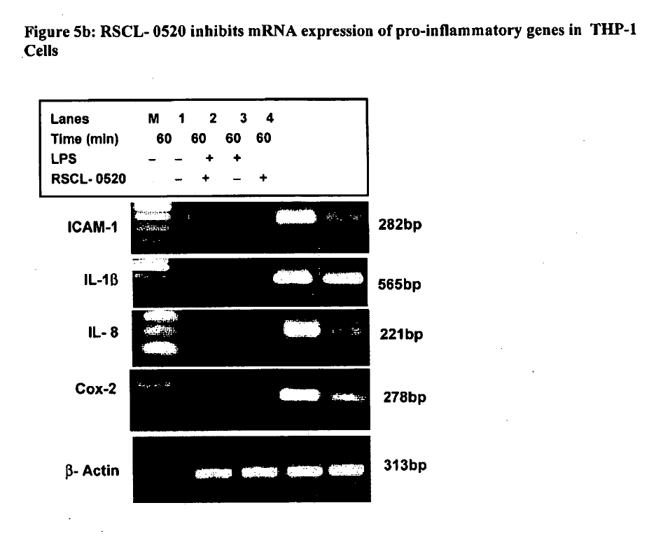

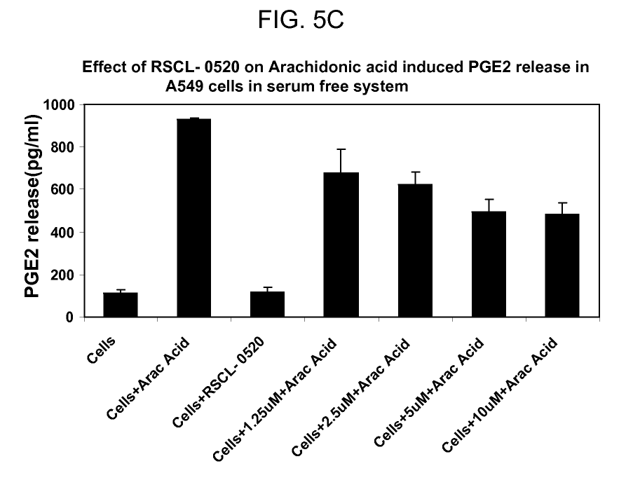

Using the comparative threshold cycle method and standard software mRNA Quantization was carried out and the data expressed as fold change of TNF-α after correction with internal control β-actin. We observed that 1 hr post LPS stimulation, RSCL-0520 down regulated TNF-α to almost control levels (FIG. 5a). On the other hand, TNF-α mRNA expression increased 7 fold after the stimulation with LPS. ***P<0.001 value is for LPS treated vs. RSCL-0520 treated followed by LPS treatment and NS is for Control Vs RSCL-0520.

Experiment 7: Effect of RSCL-0520 Inhibits mRNA Expression of Pro-Inflammatory Genes in THP-1 Cells.

The inhibitory effect of the present invention, RSCL-0520 on mRNA was checked on pro-inflammatory genes like intercellular adhesion molecule1 (ICAM-1), cyclooxygenase 2 (COX-2), IL-1β and IL-8. THP-1 cells (3×10⁶ cells) were seeded in a 6-well dish were treated with RSCL-0520 (50 μM) for 1 hr followed by incubation with or without LPS (250 ng/ml). Following two washes with ice-cold PBS, the cells were harvested and total cellular RNA was isolated using TRIZOL Reagent (Invitrogen) according to the manufacturer's instructions. cDNA was synthesised using high capacity cDNA reverse transcription kit (ABI systems). Amplification of ICAM-1, COX-2, IL-1β and IL-8 genes from the cDNA was carried out using the respective gene specific primers.

RSCL-0520 inhibited mRNA expression levels of the tested genes (FIG. 5b), indicating that its mechanism of action is via NF-κB. However, cells treated with RSCL-0520 in the absence of LPS, did not alter the gene expressions of the genes in consideration highlighting its specificity.

### Experiment 8: Effect of RSCL-0520 on Arachidonic Acid (AA) Induced PGE2 Release in A549 Cells.

COX-2 is the key enzyme regulating the production of prostaglandins, which act as central mediators of inflammation. Our earlier in-vitro data clearly demonstrated that the present invention inhibits expression of COX-2. It is well documented COX-2 pathway inhibitors were regarded as promising nonsteroidal anti-inflammatory drugs (NSAIDs). So we decided to test the ability of RSCL-0520 to block the COX-2 pathway to substantiate our earlier mRNA observation. For that purpose we chose A549 cells, a human lung cancer cell line where COX-2 is activated by AA in serum-free stimulation established by Yao et al for 12 hr. A549 cells (50×10⁴ cells/well) in serum free medium were pretreated with different concentrations (10 μM, 5 μM, 2.5 μM, and 1.25 μM) of the NSAIDs and RSCL-0520 for 30 min. (Yao J C, Duan W G, Yun Y, Liu de Q, Yan M, Jiang Z Z, Zhang L Y. Screening method for nonsteroidal antiinflammatory drugs based on the cyclooxygenase 2 pathway activated by serum-free stimulation in A549 cells. Yakugaku Zasshi. 2007; 127: 527-32.) Then the cells were incubated with AA (10M) for another 30 min. Prostaglandin E2 (PGE2), a metabolite of AA through the Cox pathway, was assayed in an enzyme immunoassay (EIA) kit from R&D systems.

The results (FIG. 5c) indicate that RSCL-0520 shows a concentration dependent inhibition of PGE2 release. RSCL-0520 is effective even at 1.25 μM enhancing its potential as potent anti-inflammatory molecule. Also, in the absence of AA, we do not see any PGE2 release clearly demonstrating that RSCL-0520 inhibits the PGE2 generated following AA stimulation.

### Experiment 9: Effect of RSCL-0520 on LPS Induced Nitric Oxide (NO) Release in RAW 264.7 Cells

In murine macrophage RAW 264.7 cells, LPS induces iNOS transcription and transduction, and then the NO production. Furthermore, LPS stimulation is well known to induce IκB proteolysis and NF-κB nuclear translocation. (Freeman & Natanson. Anti-inflammatory therapies and sepsis and septic shock. Expert Opin. Invest Drugs. 2000; 9: 165-163). Therefore, RAW264.7 cells provide an excellent model for evaluations of potential inhibitors on the pathway leading the induction of iNOS and NO production. Nitric oxide production was determined in RAW 264.7 cells from the American Type Culture Collection (Manassas, Va.) cultured in color-free DMEM with standard supplements by measuring the amount of nitrite from cell culture supernatant. RAW264.7 cells (5×10⁴ per well) were stimulated for 24 hr with or without LPS (250 ng/ml) in the absence of presence of the RSCL-0520. Nitrite, a stable end product of NO, was then measured using the Griess reaction (Green L C, Wagner D, Glogowski J, Skipper P L, Wishnok J S, Tannenbaum S R. Analysis of nitrate, nitrite, and [15-N] nitrate in biological fluids. Anal Biochem. 1982; 126: 131-38.). 100 μl of cell culture supernatant was reacted with 100 μl of Griess reagent followed by spectrophotometric measurement at 550 nm. Nitrite concentrations in the supernatants were determined by comparison with a sodium nitrite standard curve.

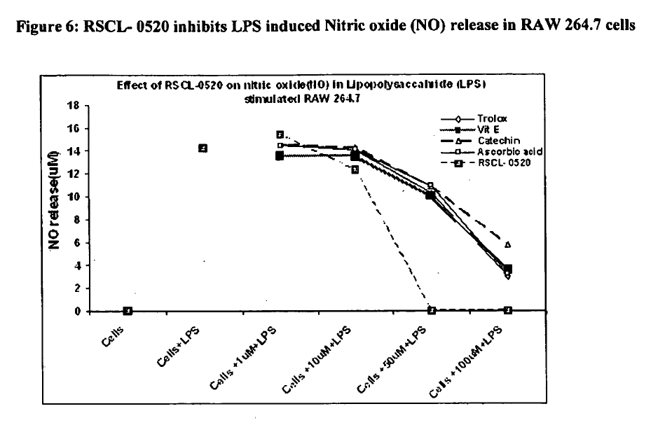

Results (FIG. 6) show that NO secretion induced by LPS stimulation was inhibited by RSCL-0520 in a concentration dependent manner. We have compared our molecule with the existing known strong anti-oxidants (Trolox, Vit E, Catechin and Ascorbic acid) and as is evident from the figure, RSCL-0520 exhibits better anti-oxidant potency than any of them. The reactive free radical NO synthesized by iNOS is a major macrophage derived inflammatory mediator and also has been reported to be involved in the development of inflammatory diseases. (Xie Q W, Cho H, Kashiwabara Y, Baum M, Weidner J R, Elliston K, Mumford R, Nathan C. Carboxyl terminus of inducible nitric oxide synthase. Contribution to NADPH binding and enzymatic activity. J Biol Chem. 1994; 269: 28500-05.). Further, it is also reported that the production of TNF-α is crucial for the synergistic induction of NO synthesis in IFN-g and/or LPS-stimulated macrophages. (Jun C D, Choi B M, Kim H M, Chung U T. Involvement of protein kinase C during taxol-induced activation of murine peritoneal macrophages. J Immunol. 1995; 154:6541-47) Our data corroborates these observations, thus clearly showing that RSCL-0520 has strong anti-inflammatory and anti-oxidant activity.

### Experiment 10: Effect of RSCL-0520 on Activation of NEMO, Degradation of IκB-α and Activation of NF-κB.

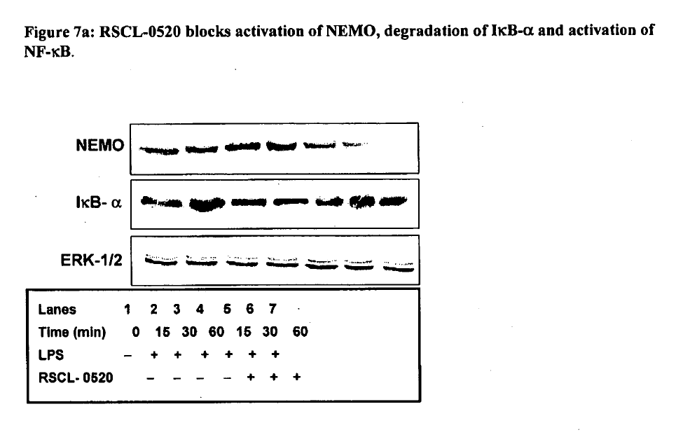

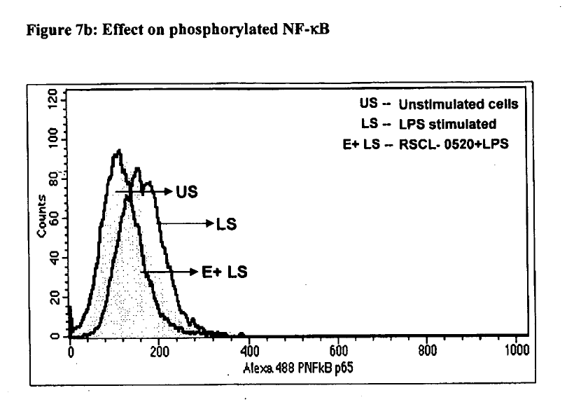

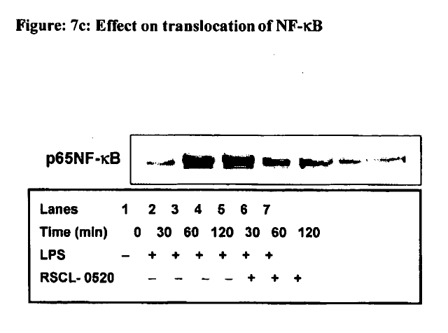

NF-κB/IκB complexes are present in the cytoplasm under unstimulated conditions. Following stimulation with LPS, we see phosphorylation and subsequent degradation of IκB allowing the free NF-κB to translocate into the nucleus to activate genes with NF-κB binding regions. Therefore, the effect of RSCL-0520 on blocking of NF-κB nuclear translocation was checked. Serum-starved THP-1 cells were stimulated with LPS (250 ng/ml) for the indicated time (FIG. 7a) in the presence and absence of RSCL-0520 (50 μM). RSCL-0520 treatment was 1 hr prior to LPS treatment. Total protein was isolated from the treated cells, and an equal amount of protein from each sample was used for immunoblots to determine protein levels of NEMO and IκB-α. Blots were stripped and reprobed using ERK-1/2 antibody to normalize the protein loading. RSCL-0520 blocked signaling to NEMO, possibly blocking phosphorylation of IKK. Which resulted in blocking p65 dissociation from IκB-α (FIG. 7a lanes 6-8). This accumulation of IκB-α lead to inhibition of subsequent down stream signaling pathways.

Further, for the detection of intracellular location of phospho NF-κB p65 subunits, 1 hr LPS-stimulated cells with and without pretreatment of RSCL-0520 (50 μM) Further processing was done as per FACS staining procedure. The cells were then stained with phospho p65 monoclonal antibody tagged with Alexa Fluor 488 (Cell Signaling Technology, Inc, Ma, USA) for 1 hr at 37° C., followed by washing. Cells were then resuspended in PBS and stained cells were acquired in BD FACS Calibur. Results (FIG. 7b) clearly show its ability to inhibit phospho-p65 in LPS treated cells, substantiating its role in NF-κB signaling inhibition.

The nuclear fractions were obtained from LPS stimulated THP-1 cells at the indicated times to evaluate the role of RSCL-0520 in inhibiting p65 subunit translocation into the nucleus. The nuclear proteins electrophoresed were processed for immunoblots using a NF-κB specific antibody. Immunoblotting profile of nuclear extracts for p65 in a time dependent manner (FIG. 6c) clearly shows that RSCL-0520 blocked translocation of NF-κB into the nucleus.

### Experiment 11: Effect of RSCL-0520 on MyD88 Dependent TLR Signaling

Serum-starved THP-1 cells were stimulated with LPS (250 ng/ml) for 1 hr in the presence and absence of RSCL-0520. Total RNA was isolated from treated cells post LPS exposure. The cDNA was used for PCR against specific primers for the TLR related genes and β-actin was used as internal control.

With the primary TLR involved identified as TLR4, we have studied the intracellular signaling accessory/adaptor molecules involved in its TLR signaling. Various adapters [myeloid differentiation primary response protein 88 (MyD88), Toll receptor IL-1R domain-containing adapter protein (TIRAP), TIR domain-containing adapter inducing IFNβ (TRIF), and Trif-related adapter molecule (TRAM)] and signaling molecules are involved in TLR-4 signaling. (Dunne A, O'Neill L A. Adaptor usage and Toll-like receptor signaling specificity. FEBS Lett. 2005; 579: 3330-35) Further, two signaling pathways; MyD88-dependent and MyD88-independent pathways have been elucidated downstream of TLR2 and 4, with MyD88 dependent pathway shown to be the most predominant one. We have shown that the present invention exerts its inhibitory effect in a MyD88 dependent manner.

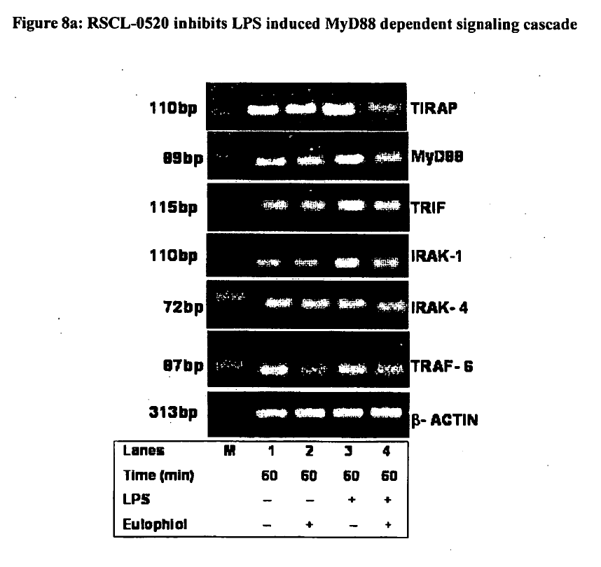

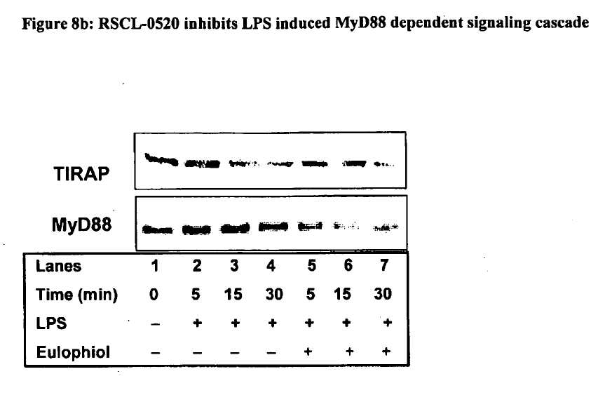

All these TLR related genes are upregulated upon stimulation with LPS (FIG. 8a lane 3); while pretreatment with RSCL-0520 (FIG. 8a lane 4) inhibits the mRNA expression levels of TIRAP, MyD88, TRAF6, IL-1R-associated kinase1 (IRAK1) and IRAK4. However, treatment of cells with RSCL-0520 did not show any effect in any of the genes at both mRNA level. These data suggest that RSCL-0520 inhibits MyD88 dependent signaling of TLR4 by LPS.

Immunoblotting further corroborated the mRNA results. Serum-starved THP-1 cells were stimulated with LPS (250 ng/ml) for the indicated time in the presence and absence of RSCL-0520. RSCL-0520 treatment was 1 hr prior to LPS treatment. Immunoblotting of total protein was carried out as before to determine protein levels of TIRAP and MyD88. Down regulation of TIRAP and also MyD88 at protein levels (similar to mRNA level) following LPS stimulation was observed in RSCL-0520 pretreated cells suggesting that RSCL-0520 inhibits MyD88 dependent TLR signaling by LPS (FIG. 8b).

### Example 12

### In Vivo Testing for Effect on LPS Induced TNF-α Release

### Experiment 1: Effect of RSCL-0520 on LPS Induced TNF-α Release in Balb/c Mice

The present invention has studied the ability of RSCL-0520 to exert protection against inflammatory agents in a mice (Balb/c) model. Balb/c (5-6 weeks) mice were injected with LPS (225 μg) intraperitoneally with and without pretreatment of RSCL-0520 (10 and 20 mg/kg). The compound was injected intraperitoneally 30 min before LPS treatment. The mice were monitored for 1 hr post LPS treatment. Untreated mice served as controls. Blood from retro-orbital plexus was collected under anesthesia 1 hr post LPS injection. Serum collected was analysed for TNF-α secretion using an ELISA detection kit.

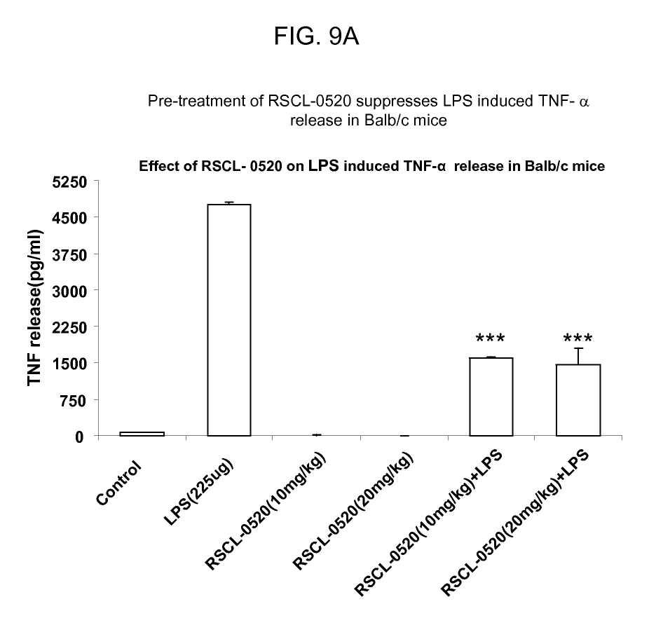

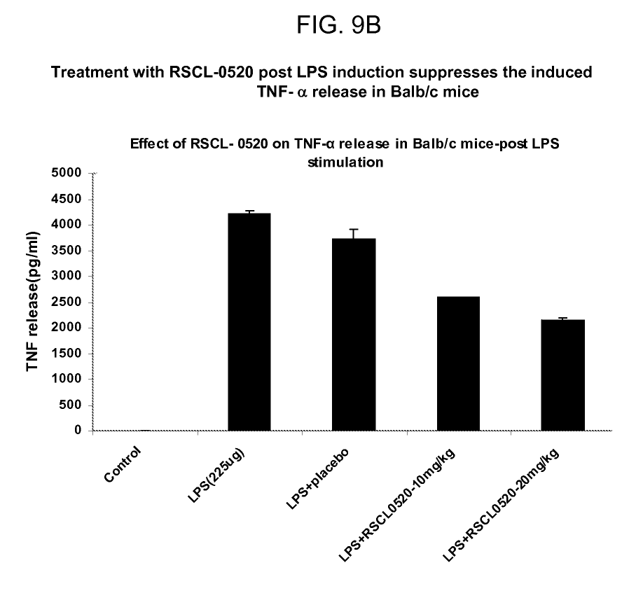

In untreated mice, LPS injection led to the secretion of large amounts on TNF-α in the serum. However, pretreated mice significantly reduced TNF production (˜66% and ˜70%) FIG. 9a) reconfirming our in-vitro data. *** P value <0.001 represents LPS treated vs. RSCL-0520 treated. The present invention shows clinical application potential to effectively attenuate LPS mediated TNF release, a key factor in various inflammatory diseases such as arthritis, sepsis and inflammatory bowel disease.

Our results have more significance from the post LPS treatment of RSCL-0520. Here we have stimulated the inflammation process in mice with an injection of LPS (225 μg) intraperitoneally and 30 min post LPS, we have challenged the ability of RSCL-0520 at two different doses (10 mg/kg, 20 mg/kg) to inhibit the TNF-α secretion. We see a dose dependent inhibition of TNF secretion (˜38% and 49% respectively, FIG. 9b.) *** P value <0.001 represents LPS treated vs. RSCL-0520 treated, NS represents non significant.

Both our in vitro and in vivo results are corroborative and clearly show our molecule to be a promising TLR antagonist and an anti-inflammatory molecule.

All publications and patent applications cited in this specification are herein incorporated by reference as if each individual publication or patent application are specifically and individually indicated to be incorporated by reference.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention.

We claim:

1. A compound of formula I: 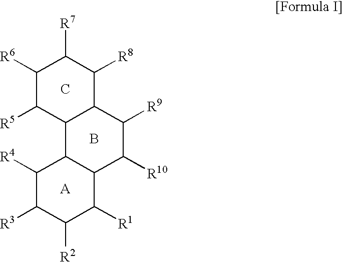
in which R¹, R², R³, R⁴, R⁵, R⁶, R⁷, R⁸, R⁹ and R¹⁰, identical or different, are a hydrogen, a hydroxy, a C₁₋₆ alkoxy, a C₁₋₆ alkyl, a halogen, a haloalkyl, a acyloxy, a hydroxyalkyl, an alkenyl, an alkenyloxy, a carboxyl, a carbalkoxy, a carbamido, a conjugated group, a substituted or unsubstituted phenyl, a substituted or unsubstituted heterocyclic group, a nitro, an amino, an acylamino, a dialkylamino, a nitric oxide (NO)-releasing moiety, wherein Ring A, Ring B, and Ring C are aliphatic or aromatic and derivatives, and polymorphs, isomers, prodrugs, pharmaceutically acceptable salts, amides and esters thereof.

2. The compound of claim 1, wherein one or more atoms are replaced with an isotope of the atom.

3. The compound of claim 1, selected from the group consisting of:
2,7-dihydroxy-3,4-dimethoxyphenanthrene (RSCL-0518);
2,7-diacetoxy-3,4-dimethoxyphenanthrene (RSCL-0519);
2,3,4,7-tetramethoxyphenanthrene (RSCL-0575);
1,5-dihydroxy-2,7-dimethoxy-9,10-dihydrophenanthrene (RSCL-0521);
1,5-diacetoxy-2,7-dimethoxy-9,10-dihydrophenanthrene (RSCL-0520);
3,4-dimethoxyphenanthrene-2,7-bis-[(2E)-3-[3,4-bis(acetyloxy)phenyl]acrylate (RSCL-0522); and
1,5-dibenzyloxy-2,7-dimethoxy phenanthrene (RSCL-0638).

4. The compound of claim 1, wherein the compound is isolated from a plant.

5. The compound of claim 4, wherein the compound is Eulophiol (RSCL-0520)

6. A pharmaceutical composition comprising a compound according to claim 1 or 3 or a salt thereof together with a pharmaceutically acceptable carrier, adjuvant or diluent.

7. The pharmaceutical composition of claim 4, for use in preparing a medicament for prevention or treatment of a disease or condition selected from the group consisting of autoimmunity, inflammation, allergy, asthma, graft rejection, graft-versus-host disease (GvHD), infection, sepsis, cancer, and immunodeficiency.

8. The pharmaceutical composition of claim 7, for use in preparation of medicaments for use in the treatment of conditions involving unwanted immune activity comprising inflammatory or autoimmune disorders.

9. The pharmaceutical composition of claim 4, comprising an effective amount of the compound for modulating immune system activity mediated by Toll-like receptors (TLRs).

10. The pharmaceutical composition of claim 9, comprising an effective amount of the compound sufficient for inhibition of TLR signaling in response to TLR ligand or TLR signaling agonist.

11. The pharmaceutical composition of claims 7 or 9, comprising an effective amount of the compound sufficient for inhibition of TLR signaling under physiological conditions.

12. The pharmaceutical composition of claim 9, comprising an effective amount of the compound sufficient for inhibiting immune stimulation via TLR antagonism.

13. The pharmaceutical composition of claim 4, wherein the compound is selected from RSCL-0518, RSCL-0519, RSCL-0575, RSCL-0521, RSCL-0520, RSCL-0638, RSCL-0522, and/or a pharmaceutically acceptable salt, hydrate, solvate, stereoisomer, amorphous solid thereof, or any combination thereof.

14. The pharmaceutical composition of claim 4, wherein the compound is an antagonist of a toll-like receptor (TLR) selected from the group consisting of TLR2, TLR3, TLR4, TLR6, TLR7, TLR8, TLR9 and TLR10.

15. The pharmaceutical composition of claim 14, wherein the compound selectively inhibits TLR4 over TLR2.

16. The pharmaceutical composition of claim 7, for use in preparation of medicaments for treatment of an autoimmune disorder selected from the group consisting of: systemic lupus erythematosus, rheumatoid arthritis, inflammatory bowel disease, Sjogren's syndrome, polymyositis, vasculitis, Wegener's granulomatosis, sarcoidosis, ankylosing spondylitis, Reiter's syndrome, psoriatic arthritis, and Behget's syndrome.

17. The pharmaceutical composition of claim 7, for use in preparation of medicaments for treating a lipopolysaccharide (LPS)-mediated disease selected from the group consisting of inflammatory bowel disease (IBD), sepsis, periodontal disease, mucositis, acne, cardiovascular disease, chronic obstructive pulmonary disease, arthritis, cystic fibrosis, bacterial-induced infections, viral-induced infections, mycoplasma-associated diseases, post herpetic neuralgia, ischemia/reperfusion injury, asthma, stroke, brain injury, necrotizing enterocolitis, bed sores, leprosy, atopic dermatitis, psoriasis, trauma, neurodegenerative disease, amphotericin B-induced fever and nephritis, coronary artery bypass grafting, and atherosclerosis.

18. An unit dose of the pharmaceutical composition of claims 7 or 9, comprising an 0.01 to 100 mg/kg of adult body weight.

19. The unit dose of claim 18 wherein the amount of the compound is selected from the group consisting of: 0.1-1.0 mg/kg body weight for inhalable dose, 0.5-10 mg/kg for oral dose, and 0.1-1.0 mg/kg for intravenous dose.

20. An article of manufacture comprising the pharmaceutical composition of claim 4 provided in a form suitable for administration to a patient in need thereof.

21. A formulation comprising (a) the pharmaceutical composition of claim 4 and (b) a second agent affecting non-antigen presenting cells bearing TLRs, in amounts sufficient to exhibit a synergistic effect on TLR-mediated immunostimulation.

22. The formulation of claim 21, wherein the second agent inhibits the proliferation of T cells.

23. A method of modulating immune system activity mediated by Toll-like receptors (TLRs) comprising contacting a cell expressing a TLR with an effective amount of a compound of Formula I 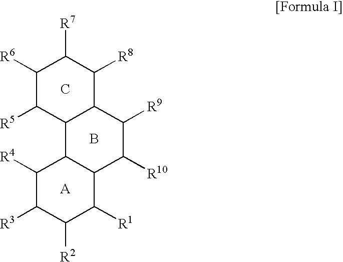
in which R¹, R², R³, R⁴, R⁵, R⁶, R⁷, R⁸, R⁹ and R¹⁰, identical or different, are a hydrogen, a hydroxy, a C₁₋₆ alkoxy, a C₁₋₆ alkyl, a halogen, a haloalkyl, a acyloxy, a hydroxyalkyl, an alkenyl, an alkenyloxy, a carboxyl, a carbalkoxy, a carbamido, a conjugated group, a substituted or unsubstituted phenyl, a substituted or unsubstituted heterocyclic group, a nitro, an amino, an acylamino, a dialkylamino, a nitric oxide (NO)-releasing moiety, wherein Ring A, Ring B, and Ring C are aliphatic or aromatic and derivatives, and polymorphs, isomers, prodrugs, pharmaceutically acceptable salts, amides and esters thereof.

24. The method of claim 23, wherein the compound is selected from the group consisting of:
2,7-dihydroxy-3,4-dimethoxyphenanthrene (RSCL-0518);
2,7-diacetoxy-3,4-dimethoxyphenanthrene (RSCL-0519);
2,3,4,7-tetramethoxyphenanthrene (RSCL-0575);
1,5-dihydroxy-2,7-dimethoxy-9,10-dihydrophenanthrene (RSCL-0521);
1,5-diacetoxy-2,7-dimethoxy-9,10-dihydrophenanthrene (RSCL 0520);
3,4-dimethoxyphenanthrene-2,7-bis-[(2E)-3-[3,4-bis(acetyloxy)phenyl]acrylate (RSCL-0522); and
1,5-dibenzyloxy-2,7-dimethoxy phenanthrene (RSCL-0638).

25. The method of claim 23, wherein the TLR-mediated immunostimulatory signaling in response to a ligand for the TLR is inhibited.

26. The method of claim 23, wherein the TLR-mediated immunostimulatory signaling in response to an agonist for the TLR is inhibited.

27. The method of claim 23, wherein TLR-mediated immunostimulation in a subject is inhibited.

28. The method of claim 23 or 24, further comprising inhibiting signaling by a Toll-like receptor (TLR) and comprising the step of contacting a cell expressing a functional TLR with an effective amount of the compound.

29. The method of claim 23 or 24, further comprising treating an autoimmune disorder selected from the group consisting of: systemic lupus erythematosus, rheumatoid arthritis, inflammatory bowel disease, Sjogren's syndrome, polymyositis, vasculitis, Wegener's granulomatosis, sarcoidosis, ankylosing spondylitis, Reiter's syndrome, psoriatic arthritis, and Behget's syndrome, by administering a therapeutically effective amount of the compound to an individual in need thereof.

30. The method of claim 23 or 24, further comprising treating or preventing a disease or condition selected from the group consisting of autoimmunity, inflammation, allergy, asthma, graft rejection, graft-versus-host disease (GvHD), infection, sepsis, cancer, and immunodeficiency, by administering a therapeutically effective amount of the compound to an individual in need thereof.

31. The method of claim 23 or 24, further comprising treating a lipopolysaccharide (LPS)-mediated disease selected from the group consisting of inflammatory bowel disease (IBD), sepsis, periodontal disease, mucositis, acne, cardiovascular disease, chronic obstructive pulmonary disease, arthritis, cystic fibrosis, bacterial-induced infections, viral-induced infections, mycoplasma-associated diseases, post herpetic neuralgia, ischemia/reperfusion injury, asthma, stroke, brain injury, necrotizing enterocolitis, bed sores, leprosy, atopic dermatitis, psoriasis, trauma, neurodegenerative disease, amphotericin B-induced fever and nephritis, coronary artery bypass grafting, and atherosclerosis, by administering a therapeutically effective amount of the compound to an individual in need thereof.

32. The method of claim 23 or 24, further comprising treating or preventing a clinical condition or disease caused by microbial pathogens, by administering a therapeutically effective amount of the compound to an individual in need thereof.

33. The method of claim 23 or 24, further comprising administering 0.01 to 100 mg of the compound per kg of adult body weight.

34. The method of claim 33 further comprising administering the compound to an individual in need thereof an amount selected from the group consisting of: 0.1-1.0 mg/kg body weight for an inhalable dose, 0.5-10 mg/kg for an oral dose, and 0.1-1.0 mg/kg for an intravenous dose.

35. The method of claim 32, wherein the individual is a veterinary animal.

36. The method of claim 32, wherein the individual is a human.

37. The method of claim 23 or 24, comprising administering the compound by a route selected from oral, intraperitoneal, intramuscular, intravenous, intra-articular, intralesional, subcutaneous, nasal, rectal, buccal, or a route sufficient to provide an amount sufficient to inhibit TLR activity

38. A method for screening agents that inhibit Toll-like receptor activation, the method comprising:
contacting a cell expressing a TLR with a candidate agent in the presence of a TLR activator or agonist; determining the effect of the candidate agent on activation of TLR.

39. The method of claim 38, wherein the candidate agent is a compound of Formula I, according to claim 1.

40. The method of claim 38, wherein the candidate agent is a phenanthrene compound.

41. The method of claim 38, further comprising comparing the activity of the candidate agent with an activity of a compound of claim 3.
